(19) United States
(12) Patent Application Publication
(10) Pub. No.: US 2009/0068162 A1
(43) Pub. Date: Mar. 12, 2009

(54) Stationary Phase Antibody Arrays for Trace Protein Analysis

(76) Inventors: Robert Sack, Brookhaven, NY (US); Ann R. Beaton, New York, NY (US); Lenard Conradi, Keil (DE); Sonal Sathe, North Bergen, NJ (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/791,045

(22) PCT Filed: Nov. 17, 2005
(86) PCT No.: PCT/US05/41735

§ 371 Date: Apr. 21, 2008

Related U.S. Application Data
(60) Provisional application No. 60/628,903, filed on Nov. 17, 2004.
(60) Provisional application No. 60/676,503, filed on Apr. 29, 2005.

(51) Int. Cl. A61K 38/43; C40B 30/04; A61P 21/00; C40B 40/10

(52) U.S. Cl. 424/94.1; 506/9; 506/18

(57) ABSTRACT

The present invention relates to the identification of trace proteins and biomarkers, e.g., TH-1/TH-2, cytokines, MMPs and angiogenic modulators in tear fluids. The present invention provides an antibody-based stationary phase array assay for simultaneously identifying, detecting and characterizing the distribution of a wide range of bioactive trace proteins in a tear fluid sample. A method for simultaneously identifying trace proteins in a biological fluid sample using a highly sensitive antibody-array assay is provided. The present invention also provides methods and kits for treating, preventing, and diagnosing ocular diseases, disorders or pathological conditions.

### FIELD OF THE INVENTION

The present invention relates to the identification of trace proteins and biomarkers, e.g., chemokines, cytokines, MMPs and angiogenic modulators, in tear fluids. In particular, the present invention relates to an antibody-based stationary phase array assay for detecting and characterizing the distribution of a wide range of bioactive trace proteins in a tear fluid sample. The invention also relates to a method of differential screening/analysis of the trace proteins in a tear fluid sample. The present invention further relates to methods and kits for diagnosing ocular diseases, disorders or pathological conditions.

### BACKGROUND OF THE INVENTION

The pre-ocular tear layer is a complex entity that contains dozens of low abundance proteins (LAPs) including many entities that are bioactive even in trace amounts. Many studies have been carried out monitoring the concentration and distribution of specific targeted LAPs in tears as a function of various parameters. This has aided in a better understanding of homeostatic and pathological processes common to the underling ocular tissue. Particular attention has been focused on those cytokines, chemokines, growth factors, angiogenic modulators and associated molecules that are known to modulate wound healing, apoptosis, cell cycling and migration.

Protein components in a biological fluid sample are involved in key physiological functions in a human individual. Keratoconjunctivitis sicca (KCS) or dry eye syndrome encompasses a diverse spectrum of diseases, which in total affect a large proportion of the population. The classification, diagnosis, characterization, pathophysiology processes and therapeutic approaches to the management of KCS have been extensively reported. One major subgroup of dry eye syndromes arises from processes attributed in part to a non Sjögren's syndrome (SS) lacrimal secretory deficiency. To identify biomarkers related to KCS progression and prognosis as well as to understand the underlying pathophysiological processes of KCS, studies have been carried out in attempt to correlate the concentration and distribution of specific proteins in the pre-ocular tear layer with the clinical symptoms of KCS. Besides the well-known general decrease in the concentrations of the constitutive lacrimal gland proteins, lysozyme, lactoferrin and certain other proteins have been identified in tear fluid which are either up-regulated or down-regulated in KCS. It has been suggested that aqueous deficient triggers an inflammatory cascade through decreased fluid turnover and increased osmolarity. The bifurcated characteristics of the tear (i.e., increased tear osmolarity and decreased in fluid turnover) may induce an epithelium related up-regulation of the production and the accumulation in the pre-ocular tear film of IL-1b and TNF-alpha. It has been hypothesized that such up-regulation results in the activation of proMMP-9 through the up-regulation of MMP-3 and thereby ultimately results in KCS.

The above hypothesis appears to be supported by certain studies of human tear fluid and animal model. However, in conducting such studies and developing methods for diagnosing and treating KCS, it has remained significantly difficult to obtain human proteomics data on tear composition due to the limited tear sample size. Until recently, virtually all studies in this field have relied either upon ELISA assays or, when appropriate, upon zymographic analysis that is carried out on either individual or, if necessary, pooled tear samples. Sample size constraints have restricted analysis in most instances to the study one or at most a few targeted proteins for a given tear sample and limited the availability of matched samples to serve as controls.

Antibody (protein) array technology offers an alternative approach for obtaining proteomic data. To this end, several laboratories have employed the enhanced multiplex analyses in the form of moving phase immuno-bead arrays coupled with flow cytometry to assay tear samples for as many as 18 low abundance proteins (LAP) in individual tear samples. Although very sensitive, such technology requires significant initial capital investment in the form of equipment and trained personnel. Moreover, at present, only a limited number of arrays have been validated for use with open eye tear fluid. Extending the range of the assay to other trace proteins and validating the methodology for use with closed eye tear fluid may be problematic. In addition, tear fluid contains “blocking factor(s)” that have been shown to interfere with the binding and the efficacy of ELISA-type assays. Although the antibody-based membrane array (MA) system has been employed to carry out differential analysis of the distribution of trace proteins in tissue extracts, tissue culture filtrates, serum and urine samples, one major disadvantage of this system is the lack of sensitivity relative to ELISA. For example, many trace proteins have yet to be identified or characterized in tear fluids by the presently available protein or antibody assay arrays.

Despite much progress, serious problems remain. One of the long-standing problems when using ELISA or multiplex assay has been the widely divergent values for the baseline levels of concentrations of many cytokines, chemokines and growth factors in the normal basal (open) eye tear fluid. It has not been uncommon to find reports of the mean concentrations for given cytokines or growth factors in the open eye tear fluid that vary by a factor of one hundred fold or more. These significant variations have made it extremely difficult to integrate the data from different studies and laboratories.

Angiogenin (ANG), which was originally identified as a tumor-derived protein, is a normal blood constituent. It was found to induce vascular growth. ANG was also reported to bind to the membrane of different cell lines and to have the ability to intervene in cellular signal transduction. Abnormality in granulocyte function can cause a subject to become susceptible to infections. ANG, including recombinant ANG, was found to inhibit the degranulation of polymorphonuclear leukocytes (PMNL), even at minute concentrations in the nanomolar (10⁻¹²) range. Although ANGs have also been implicated in tumor-associated angiogenesis, their normal physiologic function only started to be revealed recently. ANGs have been identified to be microbicidal proteins involved in innate immunity. A previously uncharacterized angiogenin, Ang4, which is produced by mouse Paneth cells, was found secreted into the gut lumen and has bactericidal activity against intestinal microbes. Furthermore, mouse Ang1 and human angiogenin, circulating proteins induced during inflammation, exhibit microbicidal activity against systemic bacterial and fungal pathogens, suggesting that ANGs contribute to systemic responses to infection. These results establish angiogenins as a family of endogenous antimicrobial proteins.

The pre-ocular tear film plays a critical major role in host defense mechanisms and homeostatic processes that differ markedly in open and closed eye environments. Studies have shown that on eye closure, inducible lacrimal flow largely ceases with ongoing flow consisting of a much slower constitutive secretion consisting primarily of secretory IgA (SIgA). This is accompanied by the recruitment of massive numbers of polymorphonuclear (PMN) cells and the induction of a sub-clinical state of inflammation. Various leukochemotactic factors including IL-8 and 12 rHETRE have been identified to accumulate in closed eye tear fluid (CTF), which drive the recruitment of PMN cells. These appear to be derived at least in part from the ocular surface tissue and may be induced by hypoxia driven up-regulation.

The present invention provides an MA system coupled with an ultra-sensitive substrate system and optimized signal-to-noise ratio that can achieve unexpectedly stable high sensitivity, e.g., one-hundred fold increased sensitivity, relative to the sensitivity obtained by the presently commercially available MA system. The present invention thus provides a facile and less expensive antibody-based MA system with high sensitivity and methods for analyzing trace protein components in a tear fluid sample.

### SUMMARY OF THE INVENTION

The present invention recognizes the successful adaptation of microwell plate and membrane antibody array technology for tear protein analysis and utilization of this technology to analyze the distribution of low abundance proteins (LAPs) in tear fluids from subjects in normal and pathological conditions (e.g., allergic or KCS basal tear fluids). The present invention is also based on the surprising discovery that ANG exists in virtually all tear samples and exhibits high levels of signal intensity in the assay array employed by the present invention.

It is an object of the present invention to simultaneously identify and analyze in one assay the trace proteins in a tear sample with sufficient sensitivity and specificity in a cost effective and rapid means.

Particularly, the present invention provides arrays to improve the sensitivity of the ELISA assays by optimizing the conditions of each assay. The present invention also discloses vast improvements of the signal-to-noise ratio by excluding from the arrays any antibody pairs that gave rise to even low levels of cross-talk between capture and probe antibodies, which allows to increase the sensitivity of assay for many LAPs (including cytokines) at least one hundred fold and thereby providing arrays suitable for the analysis of clinically obtainable size samples. The present invention also contemplates adapting a commercially available antibody array system, e.g., a 96 microwell plate formatted antibody array system, for obtaining quantitative data.

One aspect of the present invention is directed to the identification of LAPs or trace proteins in biological fluids, preferably, tear fluids. Examples of LAPs or trace proteins include, but are not limited to, matrix metalloprotease (MMP, e.g., MMPs-1, 2, 3, 8, 9, 10, 13), angiogenin (ANG, e.g., ANG-2), hematopoietic growth factor (HGF), basic fibroblast growth factor (FGFb), thromopoietin (TPO), vascular endothelial growth factor (VEGF), keratocyte growth factor (KGF), HB-epidermal growth factor (HB-EGF) and plate derived growth factor-BB (PDGF-BB), interleukins (ILs, e.g., ILs-2, 4, 5, 8, 10, 12 and 13), interferons (IFNs, e.g., IFNγ), tumour necrosis factor (TNF) (e.g., TNFα), and tissue inhibitor of metalloprotease (TIMP) (e.g., TIMPs-1 and 2). Accordingly, a method for detecting the presence and distribution of LAPs in a tear fluid sample from a subject is provided by the present invention. In a particular aspect, the method of the present invention is a qualitative method.

Another aspect of the present invention is directed to an antibody-based stationary phase array system comprising an array matrix of dot grids on a stationary phase/support/surface, preferably a membrane, bounded or attached by at least one antibody that is capable of binding with a specific protein species, secondary or detection antibodies, and an ultra-sensitive substrate that is recognized by an enzyme linked to the secondary antibodies.

Still another aspect of the present invention is directed to a method for simultaneously identifying LAPs or trace proteins in a fluid sample, preferably, a biological fluid sample, more preferably, a tear sample, comprising the steps of obtaining the sample, incubating an antibody-based stationary phase array with a blocking buffer, incubating the sample with the array, incubating the array with detection/secondary antibodies, incubating the array with an ultra-sensitive substrate that is reacted with an enzyme lined to the detection antibodies. In a particular aspect, the method can also be described to comprise the steps of: a) obtaining the sample, b) incubating the sample with an antibody-based stationary phase array comprising capture antibodies on the array, c) incubating the array from Step b with secondary antibodies, d) incubating the array from Step c with an ultra-sensitive substrate that is reacted with an enzyme linked to said secondary antibodies thereby providing a detectable signal of the binding between a capture antibody and a LAP, e) detecting the signals and analyzing data. In a particular aspect of the present invention, the method can further comprise the steps of optimizing conditions for increasing or maximizing signal-to-noise ratio, e.g., incubating an antibody-based stationary phase array with a blocking buffer prior to incubation of the sample and the array. In another particular aspect, the present invention is directed to a method for identifying trace proteins in a fluid sample, preferably, a biological fluid sample, more preferably, a tear sample, comprising obtaining the sample and assaying the sample by the antibody-based stationary phase array system of the present invention. In another particular aspect, the analysis is carried out using more than one array, e.g., three off the shelf antibody array kits, with the individual samples assayed by sequential transfer from one array to another.

Yet another aspect of the present invention is directed to a method for differential screening/analysis of trace proteins in biological fluid samples, preferably, tear samples, that are obtained from different physiological conditions or stages or status, comprising the steps of a) obtaining the samples, and b) identifying and comparing/analyzing the trace proteins in each sample.

A further aspect of the present invention is directed to a method for diagnosing pathological conditions, particularly, an ocular disease or pathological condition, of a subject, preferably, a human, comprising the steps of a) obtaining a biological fluid sample, preferably, a tear sample, b) identifying the protein distribution or level, e.g., MMP or ANG level, in the sample by the method of the present invention or by the antibody-based stationary phase array system of the present invention, and c) detecting and analyzing the changes of the trace protein distribution or level in the sample relative to that of a normal sample or to a database comprising known trace protein distribution/level patterns under normal or pathological conditions. In a particular aspect, the present invention provides a method for diagnosing an ocular infection and/or inflammations, e.g., microbial infections, including but not limited to infections related to eyes caused by bacteria, fungi or virus, or infections/inflammations caused by trauma or contact lense, or risk of susceptibility to such infections/inflammations in a subject, by detecting a varied ANG level beyond a normal range in a biological fluid sample, preferably, a tear fluid sample, from the subject.

A still further aspect of the present invention is directed to treatment of ocular infections and/or inflammation in a subject, comprising a) detecting or diagnosing an ocular microbial infection in the subject, e.g., by a detecting pathological level of ANG in a biological fluid sample, preferably, a tear fluid sample, from the subject, and b) administering ANG and/or other anti-microbial agents.

A further aspect of the present invention is directed to prevention of ocular infections and/or inflammation in a subject, comprising a) detecting or diagnosing level of ANG in a biological fluid sample, preferably, a tear fluid sample, from the subject, and b) if the ANG level is lower than its normal range, administering ANG and/or other anti-microbial agents.

One aspect of the present invention is directed to a kit for diagnosing ocular pathological conditions comprising an instruction manual, an antibody-based membrane array, a reaction-well tray, blocking and washing buffer solutions, detection antibodies, e.g., biotinylated secondary antibodies, at least one indicator that detects a specific binding of trace proteins in a test sample to the capture antibody or antibodies carried by the array, e.g., streptavidin-linked peroxidase (SPO) and a luminol-amplifier based substrate system.

Another aspect of the present invention is directed to a kit containing a composition in the form of eye drops for anti-ocular microbial infection, comprising angiogenin, preferably, recombinant angiogenin, and a pharmaceutically acceptable carrier.

### BRIEF DESCRIPTION OF THE DRAWINGS

FIG. 1A depicts Th1/Th2 well plate array assayed for OTF samples from three normal (N) individuals and an individual with active chronic rhino-conjunctivitis (CA) with one set of lanes using the LDP (top row) and the other set of lanes using the manufacturer's protocol (bottom row). Duplicate OTF samples (5 μl each) were assayed from three normals (lanes 1, 3 and 4) and from one donor with active chronic rhino-conjunctivitis (lanes 2). Arrow points to minor amount of IL-4 detected in one well using the LDP. FIG. 1B depicts array format, left to right-top row within the circle contains antibodies for IL-4, IL-5 and IL-10, middle row-antibodies for IL-8, IL-10 and IL-12, bottom row antibodies for IL-13, INFγ and TNFα. Figure C shows duplicates containing two sets of dilutions of recombinant protein standards both neat and spiked in RTF as assayed using the TH-1/Th-2 array with the LDP. Lanes 1 and 2 contain recombinant cytokine standards (IL-2 200 pg/ml, IL-4 400 pg/ml, IL-8 200 pg/ml, IL-12 200 pg/ml, IL-13 2000 pg/ml, INF gamma 200 pg/ml, and TNF alpha 800 pg/ml each) spiked in 20 μl RTF; lanes 3 and 4 contain neat standards (IL-2 200 pg/ml, IL-4 400 pg/ml, IL-8 200 pg/ml, IL-12 200 pg/ml, IL-13 2000 pg/ml, INF gamma 200 pg/ml, and TNF alpha 800 pg/ml each); lanes 5 and 6 duplicates contain 2× recombinant cytokine standard spiked in 20 μl RTF; wells 7 and 8 contain 2× standards (IL-2 400 pg/ml, IL-4 800 pg/ml, IL-8 400 pg/ml, IL-12 400 pg/ml, IL-13 4000 pg/ml, INF gamma 400 pg/ml, and TNF alpha 1600 pg/ml each) neat. Note that despite the modified protocol residual non-specific deposition still occurs at the edge of the wells that contain RTF. Blank control well plate (not shown) was devoid of significant luminescence.

FIG. 2 depicts recovery of a cocktail of recombinant cytokine spiked standards depicted in FIG. 1 C assayed using the Th1/Th2 array with the LDP.

FIGS. 3A and 3B illustrates the approximate concentration ranges of inflammatory and anti-inflammatory Th1/Th2 cytokines in RTF, OTF and CTF samples obtained from two normals (N) and an individual with active chronic rhino-conjunctivitis (CA) as assayed using the well plate array with the LDP.

FIGS. 4A-4E depict custom membrane array specific for 16 cytokines assayed for pooled OTF and CTF (30 μl) samples from two normals (N) and the corresponding control membrane. FIG. 4F depicts the array format used in the arrays of FIGS. 4A-4E. Note that in this and the other arrays the capture antibodies for several cytokines were spotted in duplicates at two or more concentrations to systematically evaluate the optimal conditions for obtaining maximal sensitivity and specificity for each assay while maintaining optimum signal to noise ratio. The number listed in the parenthesis of each of the locations on the array represents the relative concentrations of capture antibodies compared to a starting level. The effects of variation in the concentration of capture antibodies can be readily seen upon examining the signals for or IL-8 can be readily seen in this and the following two figures. Note that the positive control, which consists of a biotinylated protein, was spotted on this and the array in FIG. 5 at one hundredth the concentration that is normally found on the array in FIG. 6. This was done to avoid the signal blooming and the resultant obscuring of the adjacent weaker signals. Also note under some conditions of ultra-sensitive assay and detection the negative control as depicted in this array gives rise to a false positive signal. This possibility necessitates the use of a control membrane which in this case was incubated with an equivalent volume of a <1 kDa ultrafiltrate of RTF.

FIGS. 5B-5D depict OTF, CTF (7.5 μl) samples obtained from the same chronically allergic individual as depicted in FIG. 1A and a membrane control (incubated with an equivalent volume of the <1 kDa ultra filtrate of RTF) assayed with a different array format as shown in FIG. 5A. Note that the array configuration, the concentrations of capture antibody and the conditions of this array development differs from those utilized in FIG. 4. Under these conditions there is increased noise in the control membrane but a reduction in non-specific reactivity with the negative controls. These differences have no direct bearing upon the significance of these findings but are important in the array design.

FIGS. 6A-6C depict OTF from an individual with seasonal acerbated chronic conjunctivitis and a normal individual along with a control membrane assayed with a third array format. Note that the array configuration as shown in FIG. 6D, the concentrations of capture antibody and the conditions of this array differs from those utilized in FIGS. 4 and 5 with the concentration of the positive control not diluted resulting in blooming and a partial obscuring of adjacent signals.

FIG. 7A depicts microwell plate angiogenic array assaying normal OTF samples and protein standards using the LDP. Wellplate 1: 5 μl; wellplate 2: 2.5 μl; and wellplate 3: 1 μl of normal (N) OTF (arrow points to ANG-2); Wellplates 4, 5, 6 contain serial dilutions of recombinant protein standards. FIG. 7B depicts array format: top row-TIMP-1, ANG-2 and PDGF, middle row-TPO (can be blank see text and FIG. 7), KGF, HGF, bottom row-FGFb, VEGF and HB-EGF. FIG. 7C depicts wellplates containing (in duplicate) two dilutions of recombinant angiogenic protein standards neat and spiked in 20 μl RTF as assayed using the LDP. Wellplates 1 and 2: duplicates with angiogenic standards (TIMP-1 5000 pg/ml, Ang-2 5000 pg/ml, PDGF-bb 1,000 pg/ml, TPO 3300 pg/ml, KGF 1000 pg/ml, HGF 1600 pg/ml, HGF 1600 pg/ml, FGFbasic 2800 pg/ml, VEGF 2000 pg/ml, and HB-EGF 625 pg/ml each) in the presence of 20 μl RTF; wellplates 3 and 4: duplicates of neat angiogenic standards; wellplates 5 and 6: duplicates containing 2× angiogenic standards in 20 μl RTF: wellplates 7 and 8: neat 2× angiogenic standards. FIG. 7D depicts an assay of 5 μL OTF from normal and chronic allergic individuals (in duplicate). C: allergic samples; N: normal samples. Note that while high levels of VEGF and HGF were common to this set of samples, these growth factors were absent in many normal and pathological samples. In contrast, positive signals for FGFb and Hb-EGF were exclusive to all but one of the chronic allergic OTF samples.

FIG. 8 shows percent recovery of a cocktail of recombinant angiogenic standards assayed using an array without a TPO assay employing the LDP. Samples in duplicate consist of two concentrations of recombinant standards with and without 20 μls of a pooled RTF sample.

FIG. 9 depicts wellplates of angiogenic modulators assaying composite OTF and CTF samples (volumes as indicated) from three normal (N) donors and a donor with active chronic rhino-conjunctivitis (CA) using the LDP (Note that this array contains an assay for TPO). Donors a, b and c are normal (N) individuals (shown in FIGS. 9A-9C) while donor d has active chronic rhino-conjunctivitis (CA) (shown in FIG. 9D). FIG. 9 illustrates the range of distribution of the varying angiogenic factors in OTF and CTF samples. Note that the CTF sample from subject b was atypical compared to that from other normal individuals in that FGFb and Hb-EGF were detected. Tears for this individual are also assayed in FIG. 4 showing high levels of cytokines in CTF.

FIG. 10A depicts wellplates of MMPs assayed with a series of dilutions of RTF and CTF samples obtained from an individual with active chronic rhino-conjunctivitis (CA). Volumes of samples are as indicated. FIG. 10B depicts array format within the circle; top row-MMP-1, MMP-2, MMP-3; middle row-MMP-8, MMP-9, MMP-10; bottom row-MMP-13, TIMP-1, TIMP-2. FIG. 10C depicts serial dilution of MMP standards neat and with added RTF assayed using the MMP array. Top row-1 through 4 contain serial dilutions of MMP standards. Bottom row: control, MMP standards with and without added RTF. FIG. 10D shows 5 μl of representative OTF samples from pathological and normal individuals assayed for MMPs illustrating the range of distribution of MMPs and TIMPs in OTF. Wellplates 1-3 contain OTF samples (5 μl) from three chronic allergic (CA) individuals. Wells 4-6 contain OTF (5 μl) from three normal individuals (N). Note that arrow points to an artifact.

FIG. 11 illustrates recovery of a cocktail of recombinant TIMP and MMP protein standards at two concentrations neat and spiked in 20 μls of a pooled RTF sample assayed using the Pierce MMP array. Note that similar curves were obtained with recombinant protein standards spiked in different sources of RTF.

FIG. 12 depicts segments from four membrane arrays developed with two sets (20 μl) of OTF (recovered one month apart) after recovered from the affected and follow eyes induction of an acute unilateral allergic conjunctival reaction with samples. Samples were probed on the same array shown in FIG. 4. Array segment shows the dot ELISA arrays for IL-8 using two sets of antibody dilutions—1:2 (arrow head) and 1:5 for IL-8. Note that the difference is particularly pronounced in the 1:2 dot ELISA assays.

### DETAILED DESCRIPTION OF THE INVENTION

The present invention recognizes the successful adaptation of microwell plate and antibody array, particularly, membrane antibody array, techniques for tear protein analysis and utilization of this technology to analyze the distribution of low abundance proteins (LAPs) or trace proteins in tear fluids from subjects in normal and pathological conditions (e.g., allergic or KCS basal tear fluids). The present invention also recognizes that ANG exists in virtually all tear samples and exhibits high levels of signal intensity in the assay array employed by the present invention. The present invention discloses methods of using stationary phase forms of array analysis carry out quantitative and qualitative analysis of clinically obtainable size tear samples.

Particularly the present invention recognizes the marked difference in the pattern of distribution of various angiogenic modulators and MMPs in the normal and pathological tear samples. FGFb and Hb-EGF which are virtually absent or barely detectable in virtually all of the normal tear samples are very prominent entities in many of the dry eye tear samples, reaching concentrations which at times approaches the ng/μl range. That these growth factors are absent, or found at most in trace levels, in normal tear fluid confirms the findings of other reports. The finding of a very marked increase in Hb-EGF in the pathological tear samples is surprising since that the level of EGF was reported lower in tear fluid from individuals with both SS and non-SS aqueous deficiency dry eye syndromes. The result of the present example illustrates that Hb-EGF and EGF two closely related growth factors exhibit an inverse pattern of regulation. All of these growth factors are known to be synthesized by corneal epithelium, keratocytes, endothelium, and the lacrimal gland. Much less is known about the conjunctiva. Hb-EGF is known to secreted and bound to the corneal epithelium and other epithelial where it is found complexed with glycoproteins on the cell membrane through its heparin-binding domain. Several MMPs and other proteases are known to clip the glycoprotein releasing free HP-EGF from the cell membrane. This maybe the source of the marked increase in HB-EGF in the pathological tear samples. HB-EGF in turn is known to bind to the EGFr. Thus, chronic allergic reactions are associated with an exponential increase in the concentrations of FGFb and Hb-EGF in tear fluid. HB-EGF can be derived from HB-EGF normally bound to the epithelial cell membrane. This can be cleaved by various ADAM-like proteases including MMP-3. Released Hb-EGF can modulate apoptosis, cell migration and turnover through binding to the EGF receptor. Without intending to be limited in a particular mechanism, it is believed that FGFb can up regulate wound healing through stimulation of keratocytes.

The present invention recognizes that using both microwell plate and the membrane array techniques, OTF recovered from almost all individuals with active chronic allergic ocular surface diseases contain detectable and what is likely to be exponentially higher levels of many of the probed cytokines. According to the present invention, those cytokines that are particularly elevated include IL-2, 4, 5, 12 and INFγ. The marked increase in the levels of IL-2, 4 and INFγ confirms the premise of a strong Th2 component to chronic ocular surface allergic diseases, e.g., vernal conjunctivitis, atopic keratoconjunctivitis and giant papillary conjunctivitis. Elevated species, however, also include Th1 cytokines and encompass a mix of both activators and suppressors of inflammation. Without intending to be limited by any particular theory, it is believed that once established these pathologies represent complex cascades of events involving a multitude of pathways and the ocular surfaces has the adaptive nature to maintain homeostatic processes.

According to the present invention, the tear cytokine profiles are indicative of a chronic rather than an acute allergic reaction. Other than a modest increase in the level of IL-8 (and possibly IL-6) the cytokine profile of OTF remained largely unchanged after induction of an acute allergic reaction.

According to the present invention, the concentrations of many assayed LAPs in the pathological samples are elevated in the closed eye environment relative to the open eye environment. Without intending to be limited by any particular theory, it is believed that prolonged eye closure is associated with a marked decrease in the rate of inducible lacrimal secretion, a marked decrease in the rate of tear turnover, the induction of a sub-clinical inflammation, the recruitment and activation of PMN cells and the ensuing accumulation in CTF of a wide range of ocular surface tissue and PMN cell secretion products. Thus, the present invention contemplates detecting high levels of inflammatory mediators in the pathological CTF. The present invention also contemplates that the CTF can serve as an ideal vehicle for the recovery and detection of LAPs that are biomarkers of other ocular surface diseases because these biomarkers are much harder to identify diluted by turnover in the open eye environment.

The present invention further recognizes that the presence of antigenic species is consistent with elevated levels of f MMPs 1, 2, 3, 8, 9 and 10 in the pathological compared to normal tears especially in CTF.

One embodiment of the present invention is directed to the identification of LAPs, e.g., TH-1/TH-2 cytokines, Hb-EGF, FGF basic (FGFb), angiogenic modulators (such as ANG) and MMP, in tear fluids and the anti-microbial and an anti-inflammatory property of ANG in tear fluids. Accordingly, a method for detecting the presence of LAP, e.g., MMP or ANG, in a tear fluid sample from a subject is provided by the present invention. Without intending to be limited in a particular mechanism, it is believed that ANG exists in normal bodily fluids, e.g., blood and tears, and can function as an anti-microbial infection agent and an anti-inflammatory agent.

An object of the present invention is to simultaneously identify and analyze LAPs or trace proteins in a fluid sample, preferably, a biological fluid sample, more preferably, a tear sample, with sufficient sensitivity and specificity by a simple, cost-effective and rapid means.

According to the present invention, the stable sensitivity of an antibody-based stationary phase/support/surface array, preferably, an antibody-based membrane array (MA), can be increased, e.g., up to several-hundred fold, by modifying the array. In accordance with the present invention, a commercially available or custom-made array can be modified to maximize the sensitivity of detection, preferably, by employing an ultra-sensitive substrate, preferably, a luminol based substrate system. According to the present invention, maximizing the signal-to-noise ratio can further stabilize the high sensitivity.

Accordingly, another embodiment of the present invention is directed to an antibody-based stationary phase array system comprising an ultra-sensitive substrate, preferably, a luminol based substrate system, e.g., SuperSignal™ West Femto (Pierce). The array system of the present invention also includes, but is not limited to, an array matrix of dot grid on a stationary phase/support/surface, preferably a membrane, e.g., a Hybond nylon membrane, bounded or attached by at least one antibody, i.e., capture antibody, that is capable of binding with a specific protein species, secondary or detection antibodies that are, or can be, labeled or linked by enzymes, e.g., horseradish peroxidase (HRP), which reacts with the ultra-sensitive substrate thereby providing a detectable or visible indicator/signal of the binding between a capture antibody and a protein. For example, the detection antibodies can be HRP-conjugated secondary antibodies or biotinylated secondary antibodies that bind to HRP-conjugated streptavidin.

By a “LAP” or “trace protein” is meant a protein of minute and barely detectable amount or concentration or of very small quantity, e.g., less than 10 μg/ml in concentration. By “biological fluid sample” is meant a physical sample in liquid, fluid or mucus form that is obtained or derived from a biological source, e.g., a cell, cell culture, tissue, tissue extract, an animal or a human, which includes, but is not limited to, blood, serum, plasma, saliva, urine, tears and other bodily fluids. By “test sample” is meant a sample containing the biological fluid to be tested that is prepared for and/or carried by an antibody array.

By “negative control array/membrane” is meant an array obtained according to the array procedure in which the test sample is replaced by an appropriate mock buffer which contains substantially the same variables as the test sample except without the biological fluid to be tested. By “positive control array/membrane” is an array obtained according to the array procedure in which the test sample is replaced by an appropriate mock buffer which contains at least one, preferably, all the proteins specifically recognized by the immobilized/capture antibodies on the array.

By “differential screening” or “differential analysis” is meant a means to differentiate different trace protein distributions in test samples that are obtained, particularly from the same or similar sources, under varied physiological conditions or stages or status, e.g., from healthy and diseased subjects or at different stages of protein expression.

By “enhancing agent” is meant a chemical that can increase or improve the quality or level of detectability of a signal.

The array used for the present invention can be a standard array (e.g., RayBio™ Human Cytokine Array V (RayBiotech Inc. Norcross, Ga.)) or a variant of this array in which the concentration of the positive controls on the array is reduced, preferably, by a factor of 10.

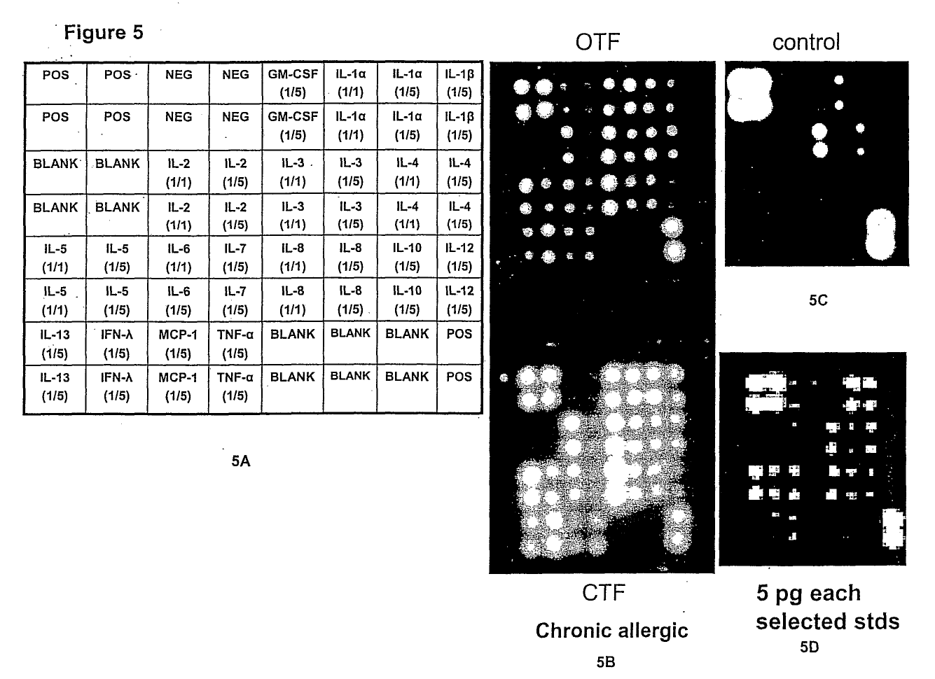

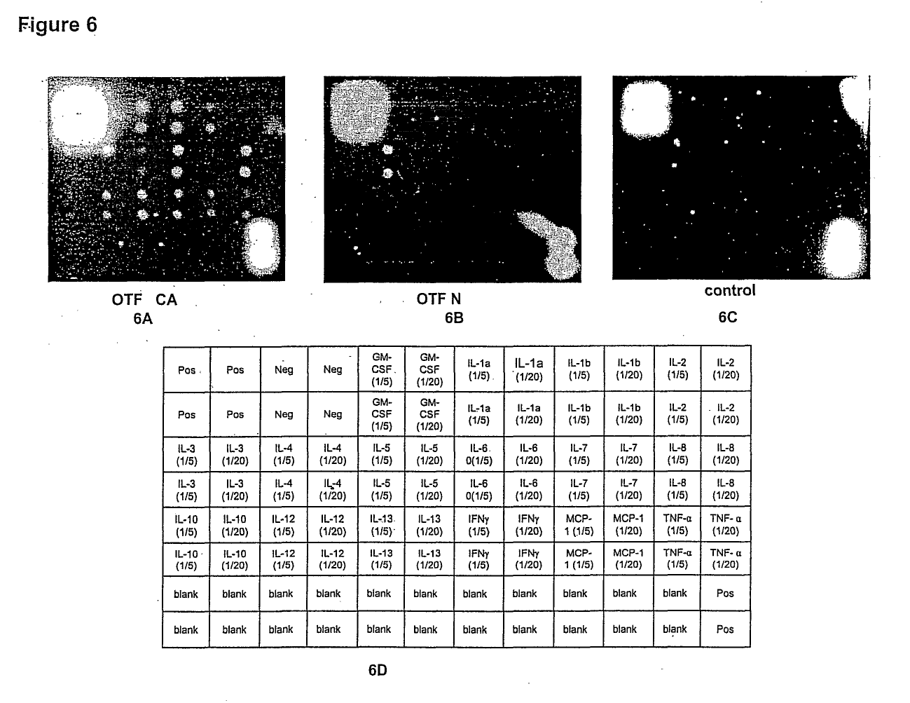

According to the present invention, the array matrix can comprise dot grids on a membrane with at least one unique capture antibody, preferably, at least one of each of a positive control, a negative control and a sample buffer control (see FIG. 6). The capture antibody or antibodies are specific for the trace protein or proteins that are contemplated to be detected. In accordance with the present invention, an array of reduced size and number of dot matrix is preferred. An example of such array is shown in FIG. 5, of which the overall matrix dimensions are reduced and the complexity of the array greatly reduced to form a 4×4 dot matrix on 12×8 mm Hybond Nylon membrane. Without intending to be limited to a particular mechanism, it is believed that background noise in the small custom array is minimal. A particularly preferred array system of the present invention is designed to allow the simultaneous screening of two or more samples for the relative distribution of more than 120 growth factors, chemokines, cytokines, angiogenic modulators and other trace proteins using a dot sandwich ELISA assay protocol.

Still another embodiment of the present invention is directed to a method for simultaneously identifying trace proteins in a fluid sample, e.g., a conditioned media sample, preferably, a biological fluid sample, more preferably, a tear sample, comprising the steps of obtaining the sample, incubating an antibody-based stationary phase array with a blocking buffer, incubating the sample with the array, incubating the array with detection/secondary antibodies, incubating the array with an ultra-sensitive substrate that is reacted with an enzyme lined to the detection antibodies.

By “ultra-sensitive substrate” is meant a substrate, particularly, a substance acted upon by an enzyme, that can be detected, at a level of femtogram (10⁻¹⁵) or attogram (10⁻¹⁸) by any chemical/biological/biochemical detection means available in the art.

According to the present invention, the standard MA kits are far too insensitive to be employed for tear analysis when they are used as directed by the manufacturer. According to the present invention, the standard assay protocol is therefore modified with the dual objectives of increasing the assay sensitivities and increasing the signal-to-noise ratio. The present invention surprisingly recognizes that the sensitivity of an MA can be increased, e.g., by several hundred fold, by substituting the supplied substrate with an ultra-sensitive substrate, preferably, luminol based substrate system, e.g., SuperSignal™ West Femto (Pierce). In a particular embodiment, the method can also be described to comprise the steps of: a) obtaining the sample, b) incubating the sample with an antibody-based stationary phase array, c) incubating the array from Step b with secondary antibodies, d) incubating the array from Step c with an ultra-sensitive substrate that is reacted with an enzyme linked to said secondary antibodies, e) detecting the signals and analyzing data.

In accordance with the method of the present invention, an antibody-based stationary phase array, preferably, a membrane array (MA), is incubated, preferably with constant mixing at an appropriate temperature, with a sufficient amount of a blocking solution of appropriate pH for a sufficient period of time. For example, a standard size membrane array, e.g., RayBio™ Human Cytokine Array V, can be incubated in 2 ml of 5% blocking grade non-fat milk (Bio-Rad) in phosphate buffered saline (PBS) at pH 7.4 for 2 hours at room temperature or overnight at 4° C. The blocking solution is then discarded and the array/membrane is incubated with sufficient volume of a biological fluid sample, for a sufficient period of time, e.g., 2 hours. For example, a standard array can be incubated with a biological fluid sample, e.g., tears, of volumes ranging from about 20 to about 200 μl brought up to a final volume of 1 ml with an appropriate blocking buffer. At least one parallel negative control array/membrane is incubated, preferably, with PBS diluted in an equivalent manner with the blocking buffer. The membrane is then washed for a sufficient time, e.g., 4 times of five minutes each with 2 ml aliquots of laboratory prepared PBS and 0.05% Tween 20 or with the manufacturer's supplied washing buffer. This is followed by a second series of additional similar washes in the same or similar type of buffer except without detergent (e.g., Tween 20). The membrane is then incubated with secondary/detection antibodies, preferably, biotinylated secondary antibodies, in a sufficient concentration and amount, e.g., the supplied cocktail of biotinylated secondary antibodies diluted to one-half of the recommended concentration in 1 ml of biotin-free casein colloidal buffer (RDI, Flanders, N.J.), for a sufficient period of time, preferably, 2 hours at room temperature or overnight at 4° C. The solution is then discarded and the washing sequence (without detergent) repeated. The array is then incubated with a sufficient amount of a blocking solution for a sufficient period of time, e.g., 2 ml of the supplied APO diluted 1:20,000 (one half the normal concentration) in the casein blocking solution for 30 minutes. The membrane is subjected to the washing sequence (without detergent) as described above. According to the present invention, for comparative analysis, matched sets of samples and control arrays are developed and imaged in tandem. For maximal sensitivity, each of the membranes is incubated with sufficient amount of the ultra-sensitive substrate, preferably, 1 ml of a freshly prepared solution of SuperSignal® West Femto (Pierce), for a short period of time, preferably, 1 minute. The membranes are then imaged by any of the well-established methods, e.g., the membranes can be stained, sandwiched between sheets of Saran Wrap and imaged using a hand luminometer (Analytical Luminescence Laboratory, San Diego, Calif.) equipped with Fuji FB-3000B film. Preferably, imaging is initiated within 5 minutes of addition of the substrate, with the film serially exposed for varying lengths of time, e.g., ranging from 10 seconds up to more than 30 minutes. In accordance with the method of the present invention, imaging continues to generate multiple images as the signal decays. As the signal decays, the difference between the samples and negative control array/membrane often becomes more pronounced.

In another particular embodiment, the present invention is directed to a method for identifying trace proteins in a fluid sample, preferably, a biological fluid sample, more preferably, a tear sample, comprising obtaining the sample and assaying the sample by the antibody-based stationary phase array system of the present invention.

In still another particular embodiment of the present invention, the method can further comprise steps of optimizing conditions for increasing or maximizing signal-to-noise ratio.

According to the present invention, it is further recognized that to improve assay sensitivity for trace proteins, the blocking process can be altered and the concentration of the biotinylated secondary antibodies optimized. In addition, membranes can be pre-treated with a sensitivity-enhancing agent, e.g., Millennium Enhancer™ used as directed by the manufacturer (BioChain Institute Inc. Hayward, Calif.).

Without intending to be limited by a particular theory, it is believed that use of an ultra-sensitive substrate system may result in background luminescence on the capture antibodies on the negative control array. This is believed to be attributed to trace levels of non-specific binding of the biotinylated secondary antibodies to the vast majority of capture antibodies as detected on the negative-control array. Therefore, according to the present invention, optimizing conditions for increasing or maximizing signal-to-noise ratio can further stabilize the high sensitivity of an assay achieved by the present invention.

According to the present invention, increasing the sample size, e.g., to several hundred μl, can optimize or increase the signal-to-noise ratio. For example, a sample size of 500 μl to 800 μl can optimize or increase signal-to-noise ratio.

According to the present invention, further increasing or maximizing the signal-to-noise ratio can achieve stable sensitivity in the method of the present invention. Thus, the signal-to-noise ratio can be improved by the partial removal from both the array and the biotinylated antibodies of cross-reacting species. For example, this can be accomplished by incubating casein-blocked membranes with the supplied cocktail of biotinylated secondary antibodies in blocking solutions. After two hours of incubation, the residual biotinylated secondary antibody solution is harvested and set aside for later use. The membranes are washed several times in PBS and then incubated for one half hour in 2 ml of 1 mM avidin (Sigma, St. Louis, Mo.) in blocking solution. This procedure serves to cap any bound biotinylated secondary antibodies (as well as the biotinylated positive controls) with avidin thereby making these species non-reactive. After washing the membrane in buffer several times to remove the residual unbound avidin, the membranes are ready for use. This process not only reduces non-specific background but also results in a decreased signal from the positive controls.

According to the present invention, a further improvement in the signal-to-noise ratio and visualization of cryptic positive entities can be accomplished by re-probing the arrays. For example, in a particular protocol, after completing SPO imaging, the arrays are incubated overnight in TRIS buffered saline (TBS) containing 0.05% Tween-20. This is followed by a series of 5-minute washes with TBS. The membranes are then incubated for 2 hours at room temperature with a 1:20,000 dilution of streptavidin-linked alkaline phosphatase (SAP) (Tropix Bedford, Mass.) in TRIS-buffered saline containing blotting-grade skim milk (BioRad). The membranes are subsequently washed five times with TRIS-buffered saline with Tween-20 followed by a second series of washes in buffer without detergent. The membranes are then re-imaged using CDP Star™ (Tropix) with the signal detected on film.

Yet another embodiment of the present invention is directed to a method for differential screening/analysis trace proteins in biological fluid samples, preferably, tear samples, that are obtained, particularly from the same or similar sources, under different physiological conditions or stages or status, comprising the steps of a) obtaining the samples, and b) identifying and comparing/analyzing the trace proteins in each sample.

Taking advantage of the methods provided by the present invention, which identify trace proteins in the amount as low as femtogram (10⁻¹⁵) or attogram (10⁻¹⁸) level, differential screening/analysis can be achieved or performed by rapidly identifying the differentially expressed or distributed proteins, particularly trace proteins in biological fluid samples from different stage or conditions, e.g., open tear fluid (OTF) and close tear fluid (CTF).

A further embodiment of the present invention is directed to a method for diagnosing pathological conditions, particularly, an ocular disease or pathological condition, of a subject, preferably, a human, comprising the steps of a) obtaining a biological fluid sample, preferably, a tear sample, b) identifying the protein distribution or level, e.g., ANG level, in the sample by the method of the present invention or by the antibody-based stationary phase array system of the present invention, and c) detecting and analyzing the changes of the trace protein distribution or level in the sample relative to that of a normal sample or a sample obtained by substantially the same or similar manner as the test sample from a normal subject, or to a database comprising known trace protein distribution/level patterns under normal or pathological conditions. According to the present invention, a normal or typical ANG level in a tear fluid sample is about 0.1 ng/ml to about 1 ng/ml, most typically, about 0.7 ng/ml.

In a particular embodiment, the present invention provides a method for diagnosing an ocular inflammation and infection, e.g., ocular infections and/or inflammation caused by bacteria, fungi or viruses or other factors, e.g., trauma or contact lenses, or the risk of susceptibility to such infections and/or inflammation in a subject, by detecting a varied ANG level beyond a normal range in a biological fluid sample, preferably, tear fluid sample, from the subject.

According to the present invention, an ocular microbial infection includes, but is not limited to, bacterial conjunctivitis, herpes simplex infection, bacterial keratitis (corneal ulcer), chlamydial and gonococcal conjunctivitis, viral conjunctivitis (pharyngoconjunctival fever and epidemic keratoconjunctivitis). Without intending to be limited by a particular theory, it is believed that the ocular ANG level, particularly the ANG level in a tear sample of a subject, increases when the subject has an ocular infection and/or inflammation. It is also believed that if the ocular ANG level, particularly the ANG level in a tear sample of a subject, is below a normal range, e.g., about 0.7 ng/ml, the subject is at risk of having, or is susceptible to ocular infections, particularly, microbial infections.

By a “normal subject” is meant a healthy subject without any detectable pathological condition by all the available medical means or a subject without a detectable particular pathological condition by all the available medical means. The contemplated pathological conditions include, but are not limited to, cancers/tumors, infections and inflammations, arterial occlusive diseases, acute myeloid leukaemia, and myelodisplastic syndromes. The contemplated ocular pathological conditions include, but are not limited to, bacterial conjunctivitis, herpes simplex infection, bacterial keratitis (corneal ulcer), chlamydial and gonococcal conjunctivitis, viral conjunctivitis (pharyngoconjunctival fever and epidemic keratoconjunctivitis).

A still further embodiment of the present invention is directed to treating ocular infections and/or inflammation in a subject, comprising a) detecting or diagnosing an ocular microbial infection in the subject, e.g., by detecting pathological level of ANG in a biological fluid sample, preferably, a tear fluid sample, from the subject, and b) administering ANG and/or other anti-microbial agents.

According to the present invention, ANG can be employed alone or in combination with one or more of other anti-microbial agents, including but not limited to, antibiotics (natural substances produced by microorganisms), synthetic antibiotics, chemotherapeutic agents (chemically synthesized), semisynthetic antibiotics (hybrid substances, which are a molecular version produced by the microbe and subsequently modified by the chemist to achieve desired properties). ANG or ANG in combination with other anti-microbial agents can be administered in oral, intravenous, or eye drop routes, or in the form of an aerosol spray.

A further embodiment of the present invention is directed to the prevention of ocular infections and/or inflammation, or having a risk of susceptible to ocular microbial infections, in a subject, comprising a) detecting or diagnosing level of ANG in a biological fluid sample, preferably, tear fluid sample, from the subject, and b) if ANG level is lower than its normal range, administering ANG and/or other anti-microbial agents.

One embodiment of the present invention is directed to a kit for diagnosing ocular pathological conditions comprising an instruction manual, an antibody-based membrane array, a reaction-well tray, blocking and washing buffer solutions, detection antibodies, e.g., biotinylated secondary antibodies, at least one indicator that detects a specific binding of trace proteins in a test sample to the capture antibody or antibodies carried by the array, e.g., streptavidin-linked peroxidase (SPO) and a luminol-amplifier based substrate system.

Another embodiment of the present invention is directed to kit containing a composition in the form of eye drops for anti-ocular microbial infection, comprising angiogenin, preferably, recombinant angiogenin, and a pharmaceutically acceptable carrier.

The present invention is illustrated by the following Examples which are not to be considered limiting the scope of the present invention in any way.

### EXAMPLE 1

### Tear Collection and Subject Selection

Tear samples were collected with informed consent according to the guidelines established by the Association for Research in Vision and Ophthalmology and the appropriate institutional review board.

Three types of tear samples were obtained from a subgroup of donors. These consisted of a reflex-type tear fluid (RTF) sample, a slowly collected basal or open eye-type tear fluid (OTF) sample and a small tear sample recovered immediately upon awaking after overnight sleep (closed eye tear fluid, CTF).

Sets of samples were recovered on an irregular basis, over a several month to a several year period, from 9 individuals who ranged in age (on onset of this study) from twenty to fifty-nine. Seven of these individuals consistently exhibited no symptoms of ocular allergies or ocular surface tissue disease while two of these individuals experienced chronic ocular allergies with acute episodes occurring during allergy seasons (beginning of spring and fall). One of these donors was diagnosed with rhino-conjunctivitis and had a clinical history that included inflamed nasal membranes and nasal polyps that required surgical intervention. The other donor had been diagnosed with seasonally enhanced atopic keratoconjunctivitis (AKC). Both individuals had undergone RAST testing with results indicating a hypersensitivity to common pollen allergens. Both donors exhibited ocular symptoms including stinging and a burning sensation that were greatly enhanced during spring and late summer, correlating with high pollen counts. Samples were divided from these two donors into those samples that were obtained during seasonal activation and those samples obtained during clinically quiescent periods.

These samples were self-collected using techniques that the present inventor has detailed elsewhere (Sack et al. Invest Opthalmol Vis Sci. 1992; 33:626-640). Briefly, RTF was collected following nasal stimulation (employing a cotton swab), with 50 μl glass microcapillaries at a rapid rate of tear flow with the total sample volume at times exceeding 1 ml. 5 μl OTF samples were collected slowly over several minutes using a calibrated 5 μl capillary with similar size tear samples recovered immediately upon eye opening after overnight sleep (CTF). Samples when available were pooled from individual donors providing sufficient volume to allow multiple analyses of the same samples.

7-10 μl sized OTF samples were also obtained in the spring from 25 normal individuals (professional school students) who had no history of recent ocular surface disease with similar samples collected, one or more times, from 7 individuals who self-reported experiencing a seasonal activation of chronic allergic conjunctivitis (these individuals to varying degrees reported ongoing year round symptoms of burning, itchy eyes with the intensity of these symptoms greatly increasing during the pollen seasons).

In addition to these samples, sets of OTF samples were obtained from a single atopic male upon laboratory provocation of an acute unilateral allergic conjunctivitis. This was accomplished by exposure of the lid to a known specific allergen (mouse nest dander). OTF samples were collected and pooled from both the fellow and the provoked eye from time zero and approximately at 15 minute intervals continuing up to two hours after initiation of the allergic reaction. This process was repeated on two other occasions separated by approximately one month apart with the acute reaction provoked in the same eye. Each set of samples were independently assayed.

Lastly, large RTF samples were recovered from several normal donors with the majority of this fluid pooled to serve as a common stock fluid. Some of this stock was used to prepare a protein-free tear ultra filtrate by passage of the sample through a 1 Kda cut off micro-centrifugal ultra-filter. This ultra-filtrate was used in the preparation of some of the controls in this study. A portion of the pooled RTF fluid was separated using preparative high-pressure liquid chromatography with the resultant molecular weight fractions concentrated by ultra-filtration. Lastly, some of the pooled RTF was used as a matrix for the preparation of spiked RTF samples that were employed in the calibration of the microwell plate assays.

All samples were kept on ice, centrifuged (11,000 rpm, 30 minutes, 40° C.) and the supernatants from a given donor, when appropriate, pooled and stored at −78° C. until analyzed.

### Saliva and Nasal Secretion Samples

Nasal secretion samples were collected by capillary tube as a by-product of induction of nasal instigation of reflex tearing. Crude sputum samples were also recovered with the samples centrifuged (11,000 rpm, 30 minutes, 40° C.) before storage.

### Microwell Plate Array Assays

Assays were carried out using a laboratory designed protocol (LDP) employing the contents of three commercially available antibody array kits (Pierce SearchLight™ Human Angiogenic Array, Pierce SearchLight™ matrix metalloprotease array and Pierce SearchLight™ TH1/TH2 cytokine array (Pierce Rockford, Ill.).

The SearchLight™ TH-1/TH-2 array is designed to simultaneously measure Interleukin (IL)s, IL-2, IL-4, IL-5, IL-8, IL-10, IL-12, IL-13, interferon-gamma (IFNγ) and tumor necrosis factor-alpha (TNFα) using volumes up to 40 μl of biological fluids. The sensitivities of each of the assays is reported to be 0.2, 0.4, 0.2, 0.4, 0.2, 0.4, 7.8, 0.2 and 1.56 pg/ml, respectively. The Pierce SearchLight™ MMP array is designed to simultaneously measure matrix metalloprotease (MMP) MMP1, MMP-2, MMP-3, MMP-8, MMP-9, MMP-10, MMP-13 and TIMP-1 and 2. While the sensitivities of each of the assays is reported to be 24, 7.8, 9.7, 24, 20, 2.4, 9.7, 8 and 1.2 pg/ml, respectively, no information was available from the manufacturer as to the molecular nature of the antigens that are used for calibration or the relative sensitivity of the assays for the pro, active and complexed forms of the MMPs. The Pierce SearchLight™ angiogenic array (as currently manufactured) is designed to allow the simultaneously assay of angiopoietin-2 (ANG-2), vascular endothelial growth factor (VEGF), heparin binding epithelial growth factor (HB-EGF)), basic fibroblast growth factor (FGFb), platelet-derived growth factor-BB (PDGF-BB), hepatocyte growth factor (HGF), keratocyte growth factor (KGF), and tissue inhibitor of metalloprotease-1 (TIMP-1) using volumes of up to 40 μl of biological fluids with the sensitivities of each of the assays reported to be 93, 12, 4, 17, 6, 10, 6, and 31 pg/ml, respectively. In the initial portion of this study an earlier version of this array was used that contained an additional dot ELISA assay for thrombopoietin-2 (TPO). Data from both versions of this array are provided with the particular version of the array identified.

All assays are based upon a classical dot sandwich ELISA protocol employing biotin-streptavidin-HRP amplification and chemiluminescence for detection. Samples were separately assayed using all three arrays. In addition as a proof of principal, pilot analysis was also carried out by successive transfer of a single set of samples and pooled mixed standards from the MMP array to the cytokine array and lastly to the angiogenic array.

Preliminary tear analysis was carried out employing the array kits as directed by the manufacturer using the instructions accompanying each of the kits (see the manufacturer's web site). This methodology has been extensively validated for use with serum, urine and tissue culture media. However, when used by both this laboratory and Pierce in house as directed for tear fluid assay, two types of matrix affects detailed below were evident that profoundly impacted the ability to obtain meaningful data. Based upon extensive studies the following laboratory designed protocol (LDP) was developed that greatly reduces the impact of both effects for most but not all of the ELISA assays.

To carry out analysis, a pre-blocking step was added to the kit protocols. This consisted of pre-incubation of the wells with 50 μl of MEGA BLOCK 3™ (a proprietary synthetic blocking agent (Cel Associates, Inc. Pearland, Tex.)) in buffer for one hour at room temperature. (This greatly decreased the extent and incidence of the aggregation of a highly sticky tear factor(s) onto the well surfaces which subsequently binds biotinylated secondary antibodies resulting in non specific reactivity). The tear samples (2-40 μl) along with a serial dilution of the supplied recombinant protein standards were then reconstituted in the same buffered blocking agent rather than the supplied sample buffer. 50 μl volumes of each of the samples were added in duplicate (whenever possible) to separate individual wells (this reduced the capacity of tear fluid to block the binding of targeted proteins to the well-bound capture antibodies). To carry out an assay, the samples were added to each well and incubated at room temperature with agitation for 60 minutes. In most instances the residual tear fluid and the standard protein fluids were decanted and discarded. In some instances the residual fluids in the wells were quantitatively harvested and stored for further use in sequential array analysis. The wells were washed six times as directed in the kits using the reconstituted supplied wash buffer. 50 μl of the supplied cocktail of the diluted biotinylated secondary antibodies was added to each well and the plate was incubated for 30 minutes at room temperature with agitation. The residual solutions were discarded and the washing sequence repeated. The wells were incubated for 30 minutes with 50 μl of the supplied streptavidin-peroxidase linked reporter enzyme with agitation, and the wells sequentially washed as directed.

Either the supplied substrate or 50 μl of freshly prepared solution of ChemiGlowTM™ (Alpha Innotech, San Leandro, Calif.) was added to each well and the wells checked for the presence of interfering bubbles. The later substrate was preferred since it had a much longer half-life. The plates were visualized using a Chemdoc XRS image station (Biorad) equipped with a deep cooled CCD camera. Imaging was carried out without binning for periods ranging from 1 minute to 10 minutes with the supplied substrate or as long as 1 hour with ChemiGlow with the background noise subtracted. The images of each of the wells in the plate were visually examined for artifactual background chemiluminescence. Most often this occurred primarily around the well edges. When affected, nearby dot ELISA assays were either excluded from analysis or whenever possible, processed with Quantity one-4.5.0™ (Biorad proprietary software) and the background chemiluminescence estimated and subtracted. Using appropriate wells with standards and spiked tear samples the same software package was used to construct two sets of standard curves: one based upon the data obtained from analysis of serial dilutions of neat recombinant protein standards and the other based upon the data obtained using the same standards spiked in pooled RTF.

To determine the effect of individual variations of the source and volume of tear fluid on the reliability and recovery of proteins in each of the dot ELISA assays, varying volumes of tear samples from different individuals were spiked with recombinant protein standards and the results of each of the ELISA assays were compared to those obtained using neat recombinant proteins standards in the absence of tear fluid. Analysis was also carried out using HPLC molecular weight fractions obtained from a pooled RTF sample.

Whenever possible tear samples were assayed in duplicate at two dilutions. Since limited volumes were available from many of the pathological samples that had to be shared for use with several microwell plate assays, these samples were often subject to a single point assay. To further conserve samples in some instances, the same samples were sequentially transferred from one array to another. This provided data suitable to obtain approximate rather than quantitative data.

### Partial Characterization of Tear Interfering Factors

Reflex tear fluid was pre-absorbed onto a variety of affinity beads with the objective of eliminating from the tear fluid interfering factors which prevented assay using the standard protocols, which is followed by centrifugation using routine protocols described by the bead manufacturers. These beads included plastic affinity beads, agarose beads linked antibodies to the heavy chain of IgA, laboratory linked antibodies to lactoferrin, lysozyme and lysine linked, and wheat germ lectin and jacalin linked agarose.

Pooled RTF samples were separated by molecular sieve high pressure liquid chromatography (HPLC) under isocratic conditions in acidic phosphate buffer on a TSK-3000 column as we have detailed elsewhere (33). Major eluting protein fractions as determined by UV absorption at 254 nm were concentrated by centrifugal ultrafiltration using appropriate cut off filters and the concentrates used for array analysis.

### Membrane Array Assays

A series of arrays were custom manufactured courtesy of RayBiotech, Inc. (Norcross, Ga.). Several of the prototype arrays were garnered in this study to serve as a proof of principal.

In the design of these experimental arrays the concentrations of the positive controls and nature and concentrations of the each of the capture antibodies were varied to contain a minimum of duplicate dots of capture antibodies specific for granulocyte-macrophage colony stimulating factor (GM-CSF), IL-1α, IL-1β, IL-2, IL-3, IL-4, IL-5, IL-6, IL-7, IL-8, IL-10, IL-12, IL-13, INFγ, monocyte chemotatic protein (MCP-1) and TNF-α. In various arrays many of the specific capture antibodies were spotted in two or more sets of dilutions. This was carried out in order to determine the optimum conditions of capture and detection for each protein and is not directly germane to this study. Dot ELISA assays were carried out using a sandwich ELISA assay employing a biotin-streptavidin amplification step and an ultra-sensitive chemiluminescent substrate for detection. Various parameters of the assay were also varied with the objective of maximizing the sensitivity of detection of individual assays while decreasing background and nonspecific interactions. To determine the sensitivity of the dot ELISA assays, arrays were calibrated using a serial dilution of recombinant protein standards (provided by Ray Biotech Inc) as well as standards from other sources. The experimental arrays that are depicted in the study allowed the detection of many of the probed protein standards well into the sub-picogram/ml range and in many instances exhibited sensitivities over one hundred fold greater sensitivity than that obtainable using standard arrays and ECL technology. The enhanced sensitivity of detection resulted in a blooming of the signal for the positive controls. To avoid this problem, in many of the arrays the concentration of positive controls was reduced 1/100 fold that of the concentration employed on current commercial products. Many array and assay parameters were modified in this broad study only a few specific arrays were chosen for purposes of illustration of specific phenomena. Given this variability a general protocol is described as follows, which is by no means to limit the scope of the present invention in any way.

The arrays were incubated in 2 ml of 5% biotin-free casein colloidal buffer (RDI, Flanders, N.J.) in phosphate buffered saline (PBS) at pH 7.4. This and all subsequent incubations were carried out with constant rocking at room temperature. After two hours, the blocking solution was discarded and the membranes were incubated with the tear samples (5 to 30 μl) that had been brought up to a final volume of 1 ml in the presence of the above blocking buffer. Each array was assayed with a parallel negative control array incubated either with PBS or in some instances with a <1 Kda ultrafiltrate from RTF diluted in an equivalent manner with the blocking buffer. In some instances, arrays were run in parallel with recombinant protein standards spiked in RTF.

After two hours of incubation with tear or other samples, the membranes were washed three times for five minutes each with 2 ml of laboratory prepared PBS and 0.05% Tween 20 followed by a second series of three washes for five minutes each in PBS buffer without detergent. The membranes were then incubated with the supplied cocktail of biotinylated-secondary antibodies. This was diluted to one-half of the recommended concentration in 1 ml of biotin-free casein colloidal buffer. After incubation for 2 hours at room temperature, the solution was discarded and the washing sequence repeated. The membranes were then incubated with 2 ml of the supplied horse radish peroxidase (HRP) conjugated streptavidin diluted 1:20,000 (one half the normal concentrations) in the casein blocking solution. After one half-hour, the residual fluid was discarded and the membranes subjected to a final washing sequence.

For comparative analysis, matched sets of samples and control arrays were developed and imaged in tandem. For maximal sensitivity, each of the membranes was incubated with 1 ml of a freshly prepared solution of ECL Advance Western Blotting Detection Kit (Amersham Biosciences, Piscataway, N.J.) for 1 minute. The membranes were stained, sandwiched between sheets of Saran Wrap and imaged using a hand luminometer (Analytical Luminescence Laboratory, San Diego, Calif.) equipped with Fuji FB-3000B film. Imaging was initiated after 1 minute of addition of the substrate, with the film serially exposed for varying lengths of time ranging from 10 seconds up to more than one half hour. As the signal decayed, the difference between the samples and negative control membrane often became more pronounced. Imaging was also accomplished in a Biorad ChemiDoc XRS image station equipped with an enhanced sensitivity −45° C. cooled-backed 12-bit CCD with a dynamic range >3.

### Results

### Microwell Plate Array Assays—Tear Matrix Effects and the Development of a LDP

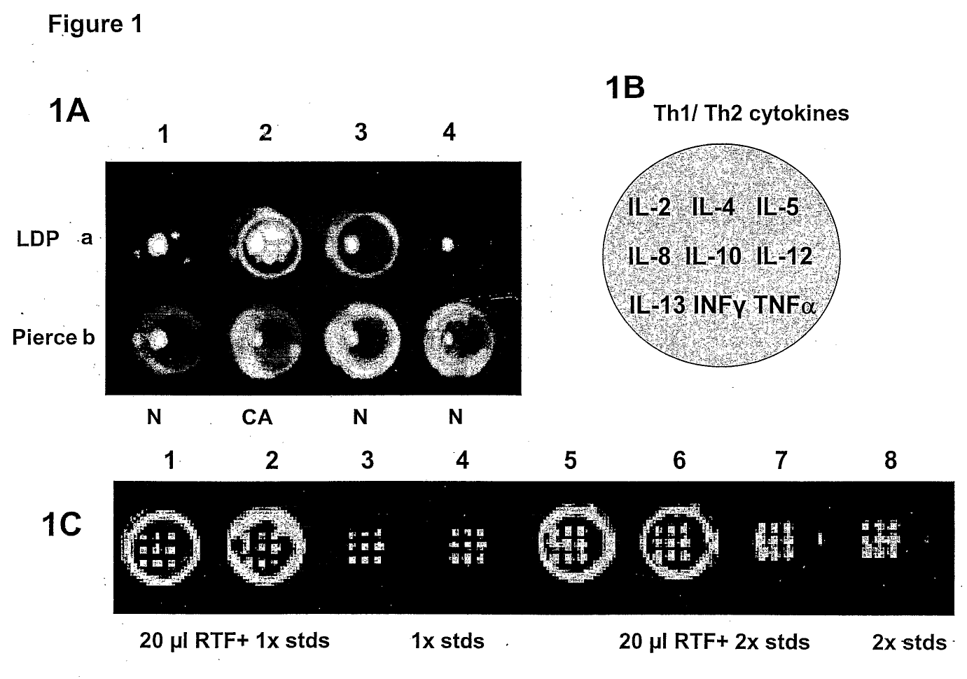

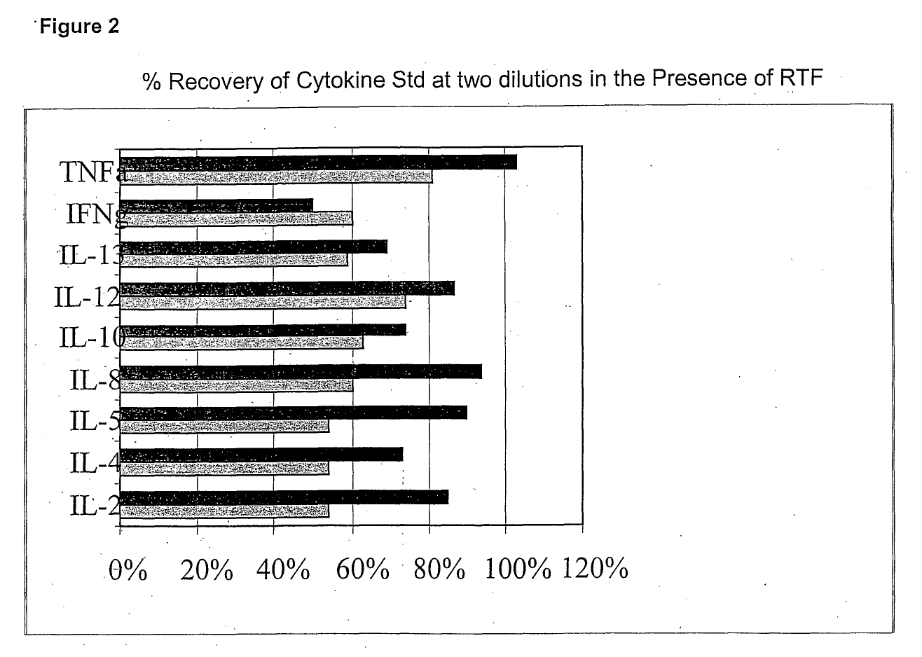

When tear samples were assayed using the Pierce SearchLight™ array kits as configured without any modification, two tear specific matrix effects were evident that profoundly limited the capacity to obtain meaningful data. These consisted of a high level of non-specific background chemiluminescence (FIG. 1A-row b) and the partial to complete obstruction of the capacity to detect recombinant protein standards spiked into tear fluid (not shown). The latter process also resulted in the presence in tear fluid of cryptic non-reactive pools of targeted proteins (FIG. 1A-rows a and b as an example).

Non-specific background chemiluminescence was most evident when assaying larger sized samples (RTF and OTF) using the more sensitive cytokine array. This artifact can be attributed to the presence in tear fluid of a highly sticky factor(s) or complex that exhibits a predilection for the microwell plastic. This substance preferentially aggregates around the well edges and subsequently binds the biotinylated secondary antibodies resulting in non-specific background chemiluminescence. A similar phenomenon could be observed on the assay of sputum but not nasal secretions.

Passage of RTF through a molecular sieve HPLC column resulted in an eluent largely devoid of aggregating activity when the eluted fractions assayed either separately or re-combined (not shown). This would suggest that the interfering factor(s) either represents a dissociable complex or that it is retained on the column.

Attempts to reduce or eliminate these artifacts by selectively removing the interfering factors from tear fluid prior to assay by the pre-incubation of tear fluid with surface active plastic beads, or by precipitation in the presence of commercially prepared lectin (jacalin, wheat germ agglutinin), protein L, IgA and IgG specific-beads and laboratory prepared lysozyme and lactoferrin specific beads all proved unsuccessful. Pre-incubation of the microwell plates, however, with various blocking agents including I-Block™ (Tropix Bedford, Mass.), Casein (Bio-Rad), Bovine serum albumin and fish gelatin and inclusion of these blocking substances in the sample incubation buffer to varying degrees, reduced the impact of both matrix effects. Best results were obtained incorporating MEGA-BLOC 3™ (a proprietary pre-buffered synthetic blocking agent) in a LDP protocol (see above). This allowed for the assay of up to 30 μl of OTF, while greatly reducing the risk and extent of non-specific background chemiluminescence and allowing for the detection of previously cryptic pools of targeted proteins (Compare FIG. 1A top and bottom rows).

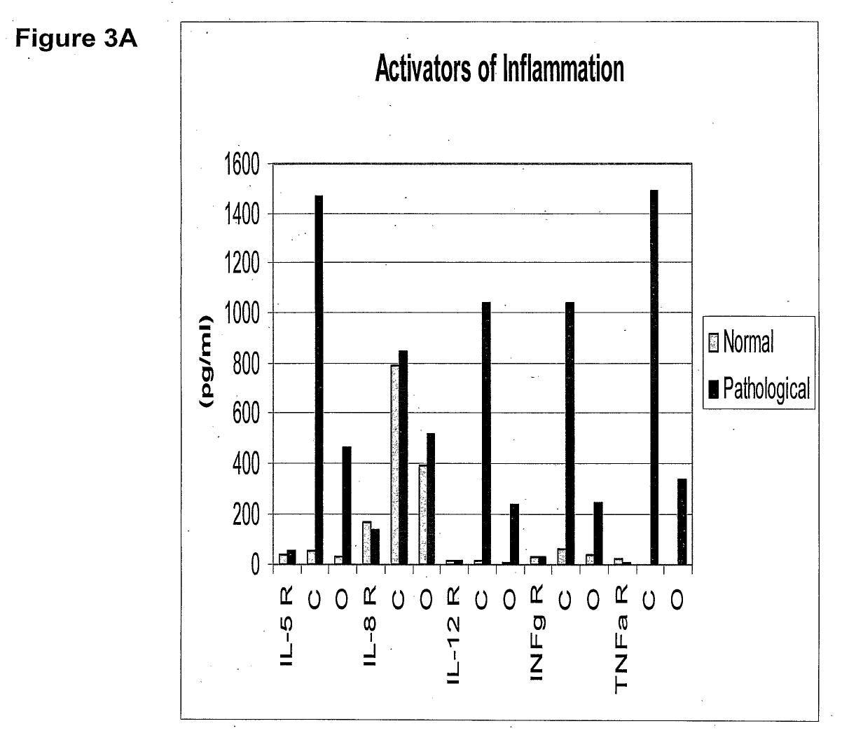

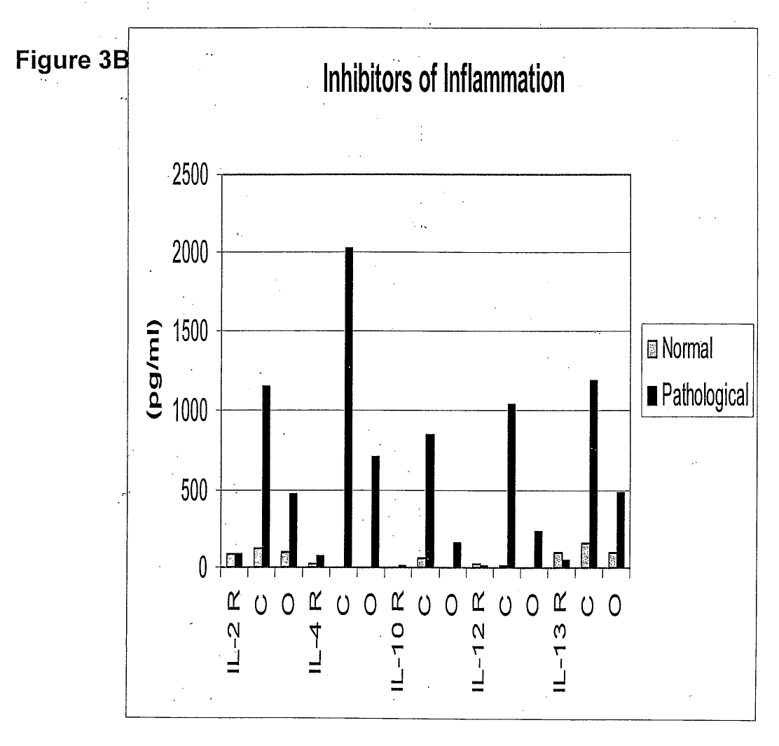

Significantly, irrespective of the choice of blocking agents or the conditions of assay, neither matrix effects could be totally eliminated. Low levels of non-specific aggregation still occurred occasionally while the percent recovery of spiked proteins proved quite variable (FIGS. 3, 7, 8, 10 and 11). In most instances, however, the residual non-specific chemiluminescence was restricted to the well edge. Since this often is geographically separate from the dot ELISA assays, by carefully avoiding the integration of the luminescence of the well edges it often proved possible to salvage the data from individual affected wells. In those rare instances where non-specific deposition led to a relatively uniform background, this could be subtracted (using the Bio-Rad proprietary software) thereby allowing the approximation of the concentration of the probed proteins. For the above reasons analysis requires the visual inspection of each of the wells in the plate prior to computerized densitometry analysis. Quantitative estimations were based upon standard curves constructed from assay of recombinant protein standards in the presence and absence of added RTF with the percent recovery of protein standards spiked in RTF used for correction (see FIGS. 3, 7 and 10).

Since the extent of interference with the recovery of spiked protein standards was found to vary with the size and nature (RTF, OTF and CTF) of the tear sample, the results in this study can at best be viewed as semi-quantitative rather than quantitative in nature.

### Microwell Plate Assay of Cytokines

Assay of spiked tear samples with the TH-1/TH-2 array and the laboratory designed protocol (LDP) allowed for the recovery of ˜60-80% of each of the recombinant cytokine standards (FIG. 1B and table II) and revealed the cytokine profiles of the OTF samples from the normal and chronic allergic populations to be decidedly different.

The normal OTF profile invariably exhibited a moderate to strong signal for IL-8, which was occasionally accompanied by much lower and more variable signals for IL-4, TNFα or other cytokines (FIG. 1 A-row a and FIG. 3). In contrast, the OTF samples from all but one of the pathological population (6 of 7 individuals) exhibited extremely intense signals for Il-2, Il-4, IL-8, IL-10, II-12 and TNFα. These signals were far more intense and the differential was far greater when contrasting the CTF rather than OTF samples (see FIG. 3). In longitudinally collected pathological OTF samples, the level and distribution of cytokines was found to parallel the changes in clinical symptomology with the cytokine profile approaching that of normal tear fluid during the quiescent clinical periods.

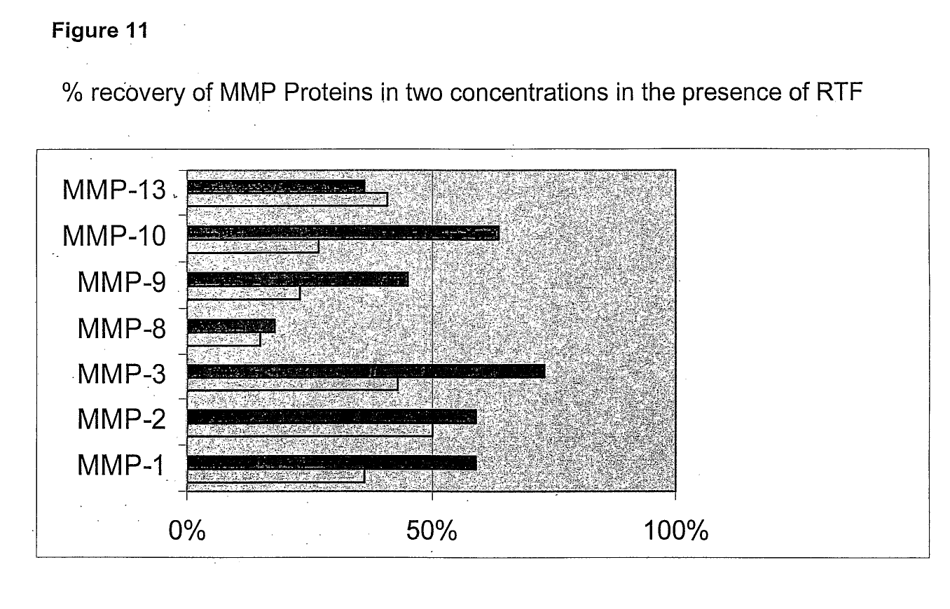

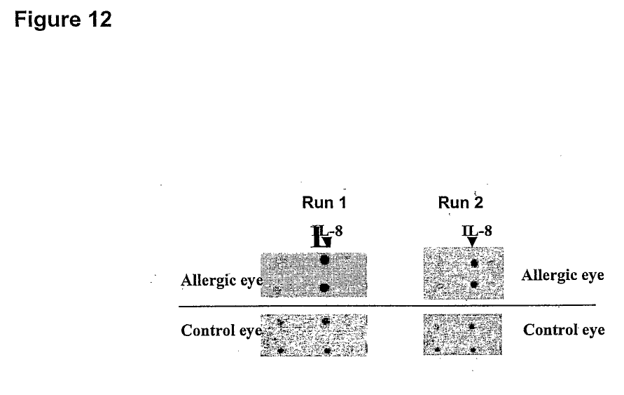

In contrast to OTF collected during chronic allergic condition, OTF recovered subsequent to the induction of an acute monocular atopic reaction proved strikingly similar to that of the control eye exhibiting only a marginal increase in the level of IL-8 and possibly IL-6 (FIG. 11).

### Membrane Array Analysis of Cytokines

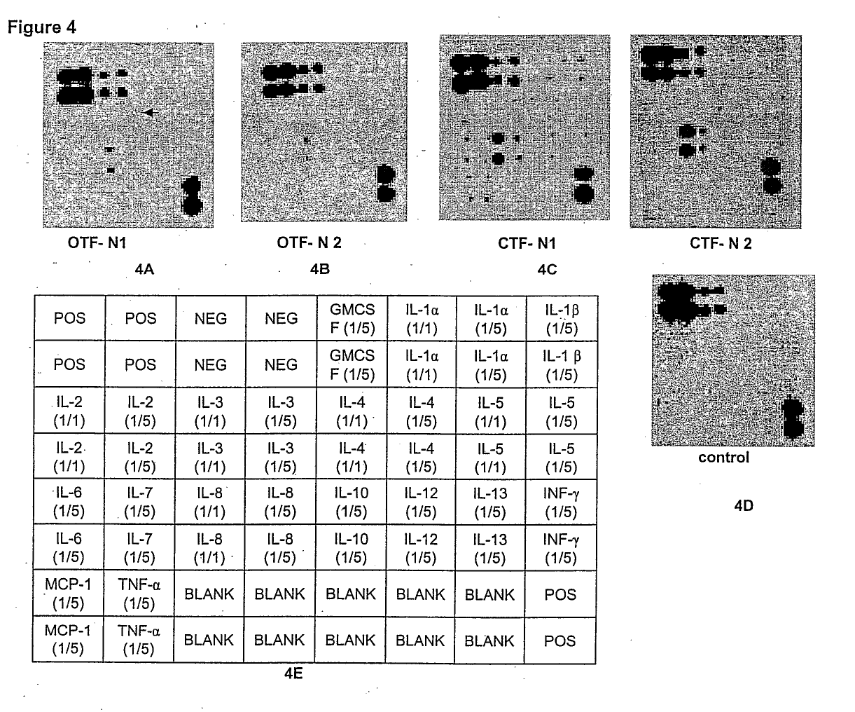

A number of experimental membrane arrays were manufactured courtesy of RayBiotech with the objectives of minimizing the extent of cross-reactivity between capture and probe antibodies, and optimizing the conditions of each assay and increasing the signal-to-noise ratio. Under optimum conditions the sensitivity of the individual dot ELISA assays was increased in some instances over one e hundred fold or more over that obtained with conventional ECL assays thereby allowing the visualization of many cytokines in concentrations well down into the femtogram range (FIG. 5). This allowed for the differential assay and the determination of the relative distribution of all 16 of probed cytokines and inflammatory mediators on the arrays using small pooled OTF (30-15 μl) samples as well as individual (˜5-8 μl) CTF samples obtained from the normal and pathological populations (FIGS. 4-6). Note that these arrays contained capture antibodies for many probed proteins in two or more dilutions. This was done in order to assess the effect of variation in the concentration of capture antibody upon the visualization of the targeted protein and non-specific cross reactivity as seen in the negative control membrane. The latter factor highlights the critical importance of running a parallel control membrane when using this ultra sensitive assay protocol. The specific results parallel and dramatically expand upon the findings of the microwell plate assays revealing markedly elevated levels of virtually all 16 of the probed proteins in the pathological samples. As anticipated the intensity and difference in signals proved far more extreme when contrasting CTF to OTF (FIGS. 4-6).

### Angiogenic Array Assays

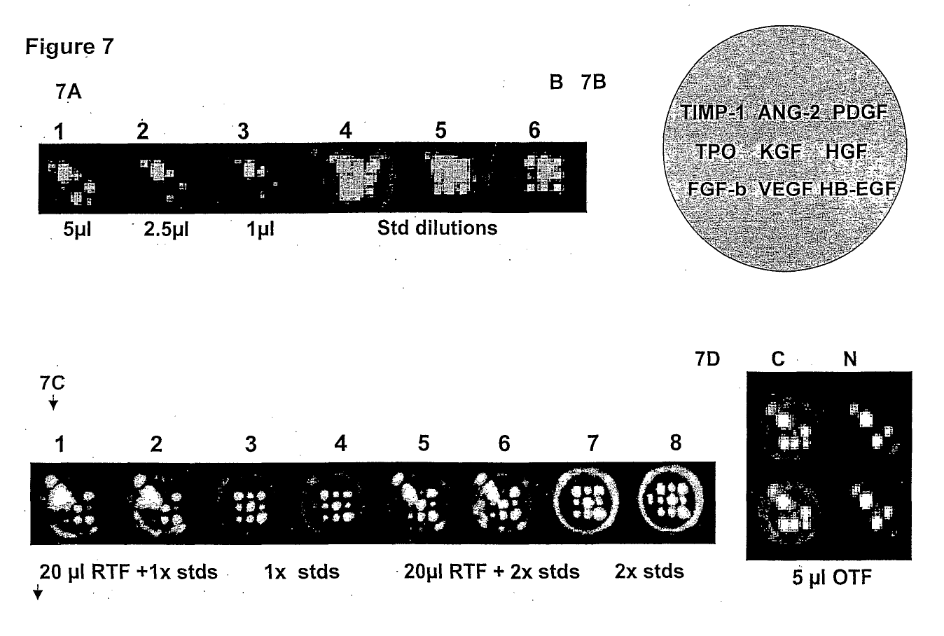

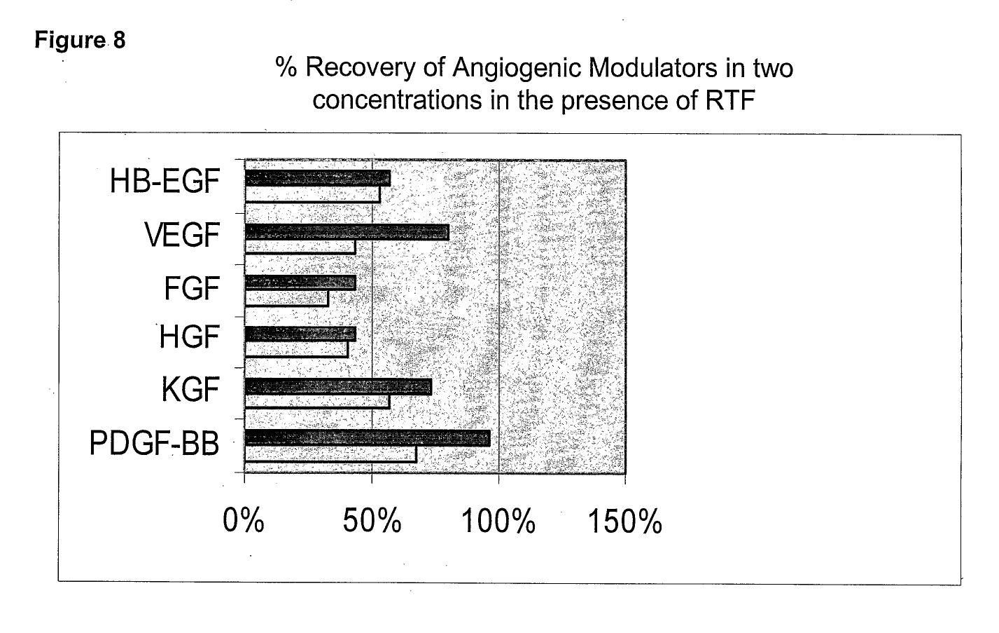

SearchLight angiogenic array assay of spiked RTF samples with the LDP revealed very pronounced differences in the levels of recovery of specific protein standards (FIG. 7 row A and FIG. 8). Virtually all of the OTF samples exhibited very intense signals for TIMP-1. Within the sample size selected for assay (10-20 μl) the signal for TIMP-1 often exceeded the linear range of the assay. A minority of both the normal as well as pathological OTF also exhibited strong signals for VEGF and less frequent and less intense signals for HGF (FIG. 8). OTF samples from all but one individual with chronic allergic conjunctivitis exhibited strong to very intense signals for HGFb and HB-EGF, two growth factors that were virtually absent in all of the OTF samples from the normal population (FIG. 6 D). Moreover, longitudinal analysis revealed that the presence and levels of HGFb and HB-EGF in OTF paralleled the clinical course of disease waxing and waning of the clinical symptoms. In contrast, neither of the growth factors could be detected in OTF obtained after instigation of a unilateral acute allergic reaction.

Comparative analysis of OTF and CTF samples from the normal and the chronic allergic populations revealed far more intense signals in the CTF compared to OTF samples with the CTF sample from one asymptomatic donor also exhibiting signals for EGFb and HB EGF (FIG. 8).

### MMP Microwell Plate Array Assays

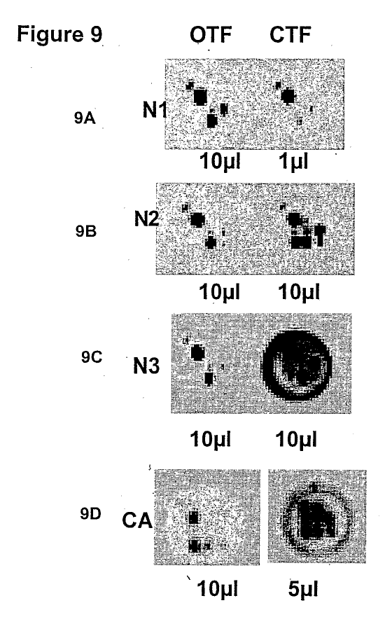

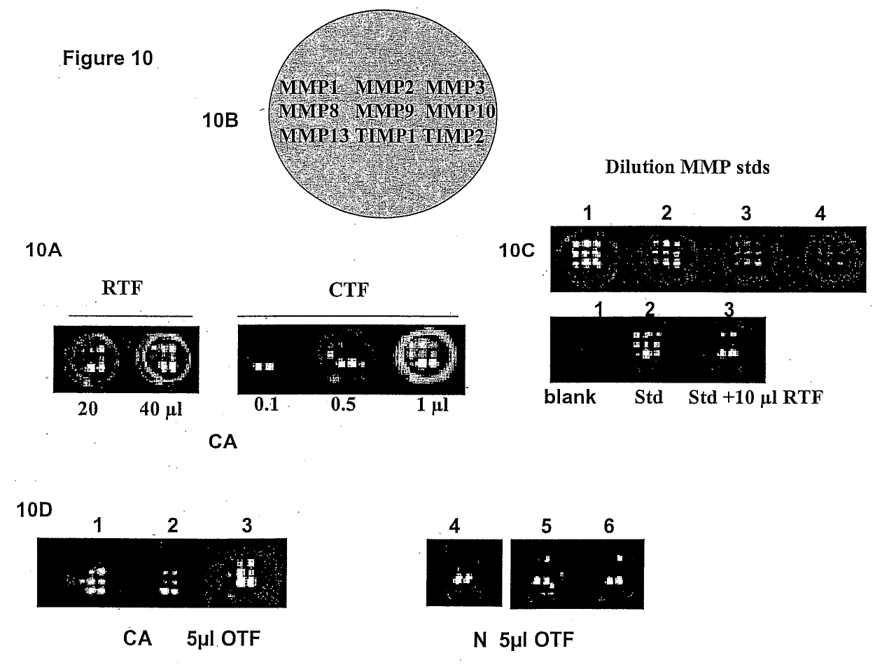

Comparative assays of TIMPs and MMP species using the Pierce array with the LDP reveals a low level of recovery for many of the MMP (MMP-1, 8, 9 10) standards when spiked in RTF samples (FIGS. 9 and 10). It is believed that RTF can contain high levels of various entities (i.e. inhibitors or receptors) that can complex or transform these spiked protein standards converting them into antigenically non-reactive entities.

Differential analysis of OTF and CTF obtained from the normal and the pathological populations, however, revealed striking differences in the patterns of distribution of detectable signals. OTF samples from normals exhibited intense signals for TIMPs 1 and 2, but at most only trace signals for MMP-3 and/or MMP-10. In contrast, OTF samples from virtually all of the allergic individuals exhibited intense signals for a variable mix of MMP species including MMP-1, 2, 3, 8, 9 and 10 (FIG. 9). As anticipated the array profile for OTF and CTF proved strikingly different with the latter samples exhibiting far more intense signals for both TIMP 1 and 2, as well as strong signals for several MMP species including MMPs 1, 2, 8 and 9 (FIG. 9).

### EXAMPLE 2

### Tear Collection

Tear samples were routinely recovered over a several month period from 6 normal male and female subjects, who ranged from twenty-five to fifty-nine years of age. Reflex tear fluid (RTF) was collected following nasal stimulation using a 50 μl glass capillary tube at a rapid rate of tear flow. Open eye (basal) tear samples (OTF) were collected slowly over a several minute period using a 5 μl calibrated glass microcapillary tube. Immediately upon eye opening after overnight sleep, similar size closed eye tear samples (CTF) were collected. Samples were transported to the laboratory on ice and centrifuged (11,000 rpm, 30 minutes, 4° C.) with the RTF, OTF and CTF supernatants from each donor separately pooled and stored at −78° C. until analyzed.

### Membrane-Micro Array Assays

The majority of the work was carried out either with a standard array (RayBio™ Human Cytokine Array V (RayBiotech Inc. Norcross, Ga.)) or a variant of this array in which the concentration of the positive controls on the array was reduced by a factor of 10. The array matrix consisted of an 11 by 8 dot grid on a 20×30 mm Hybond membrane with 79 unique capture antibodies, 6 identical positive controls containing a biotinylated protein standard and three negative controls consisting of two dots of Bovine Serum Albumin (BSA) and one dot of the sample buffer. The capture antibodies were specific for Angiogenin (ANG), B-lymphocyte chemoattractant (BLC), Brain-derived neurotrophic factor (BDNF), Chemokine-beta-6 (Eotaxin-2), Chemokine-beta-8-1 (Ck beta 8-1), Serotoxin (Eotaxin), Epidermal growth factor (EGF), Epithelial neutrophil-activating protein 78 (ENA-78), Fibroblast growth factor-4 (FGF-4), Fibroblast growth factor-6 (FGF-6), Fibroblast growth factor-7 (FGF-7), Fibroblast growth factor-9 (FGF-9), Fractalkine (FKN), Fms-like tyrosine kinase-3 ligand (Flt-3 Ligand), Glial-derived Neurotrophic Factor (GDNF), Granulocyte Chemotactic Protein 2 (GCP-2), Granulocyte-colony Stimulating Factor (GCSF), Granulocyte-macrophage colony stimulating factor (GM-CSF), Growth Related Oncogene (GRO), Growth Related Oncogene-Alpha (GRO-α), hematopoietic growth factors, hepatocyte growth factor (HGF), I-309 (I-309), IFN-γ Inducible Protein 10 (IP-10), Insulin-like growth factor 1 (IGF-1), Insulin-like growth factor binding proteins 1 (IGFBP-1), Insulin-like growth factor binding proteins 2 (IGFBP-2), Insulin-like growth factor binding proteins 3 (IGFBP-3), Insulin-like growth factor binding proteins 4 (IGFBP-4), Interferon gamma (IFN-gamma), Interleukin 1 Alpha (IL-1a), Interleukin 1Beta (IL-1b), Interleukin 2 (IL-2), Interleukin 3 (IL-3), Interleukin 4 (IL-4), Interleukin 5 (IL-5), Interleukin 6 (IL-6), Interleukin 7 (IL-7), Interleukin 8 (IL-8), Interleukin 10 (IL-10), Interleukin 12 (IL-12), Interleukin 13 (IL-13), Interleukin 15 (IL-15), Interleukin 16 (IL-16), Leptin (Leptin), LIGHT (LIGHT), Leukemia inhibitory factor (LIF), Macrophage Inflammatory Protein 1Beta (MIP-1b), Macrophage Inflammatory Protein 1Delta (MIP-1d), Macrophage Inflammatory Protein 3Alpha (MIP-3 a), Macrophage-Colony Stimulating Factor (MCSF), Macrophage-derived Chemokine (MDC), Mesoderm inducing factor (MIF), Monokine induced by gamma interferon (MIG), Monocyte Chemoattractant Protein 1 (MCP-1), Monocyte Chemoattractant Protein 2 (MCP-2), Monocyte Chemoattractant Protein 3 (MCP-3), Monocyte Chemoattractant Protein 4 (MCP-4), Neutrophil Activating Peptide 2 (NAP-2), Neurotrophin-3 (NT-3), Neurotrophin-4 (NT-4), Oncostatin M (OSM), Osteoprotegerin (OPG), Placenta growth Factor (PIGF), Platelet-derived Growth Factor-BB (PDGF-BB), Regulated-upon activation, normal T-cell expressed and presumably secreted (RANTES), Pulmonary and Activation-Regulated Chemokine (PARC), Stem Cell Factor (SCF), Stromal cell-derived factor (SDF-1), Thrombopoietin (TPO), Thymus and Activation-Regulated Chemokine (TARC), Tissue inhibitor of metalloproteinases-1 (TIMP-1), Tissue inhibitor of metalloproteinases-2 (TIMP-2), Transforming growth factor beta 1 (TGF-beta 1), Transforming growth factor beta 2 (TGF-beta 2), Transforming growth factor beta 3 (TGF-beta 3), Eotaxin-3, Tumor necrosis factor-alpha (TNF-a), Tumor necrosis factor-beta (TNF-b) and Vascular Endothelial Growth Factor (VEGF).

The second array that was used in this Example was a prototype provided as a gift of the manufacturer (RayBiotech Inc.). The overall matrix dimensions were reduced and the complexity of the array greatly reduced to form a 4×4 dot matrix on 12×8 mm Hybond Nylon membrane. This matrix consisted of three positive controls (at one-tenth the standard concentration), one negative control, and 12 capture antibodies. The capture antibodies were specific for ANG, ENA-78, Eotaxin, FGF-7, IL-8, TIMP-1, VEGF, TNF-a, IGFBP-3, OSM and NT-3 and a previously unprobed protein, Angiopoietin-2 (APO-2). The array composition was selected in part to provide qualitative data to complement quantitative data obtained by micro-well plate formatted array assays data (Pierce SearchLight Array™).

A standard size array was processed in the manufacturer supplied well-plate chambers. A mini-array was developed in smaller chambers of a 16 well tissue culture micro-well-plate thereby allowing a 50% reduction in the volumes of all of the added solutions. The standard size arrays were incubated in 2 ml of 5% blocking grade non-fat milk (Bio-Rad) in phosphate buffered saline (PBS) pH 7.4. This and all subsequent incubations were carried out with constant rocking at room temperature. After two hours, the blocking solution was discarded and the membranes were incubated with the tear samples (volumes ranging from 20 to 200 μl) brought up to a final volume of 1 ml with the blocking buffer. A parallel negative control membrane was incubated with PBS diluted in an equivalent manner with the blocking buffer. After two hours of incubation with tear samples, the membranes were washed 4 times (for five minutes each) with 2 ml aliquots of laboratory prepared PBS and 0.05% Tween 20 or with the manufacturer's supplied washing buffer. This was followed by a second series of four additional five minutes washes in buffer without detergent. The membranes were then incubated with the supplied cocktail of biotinylated secondary antibodies that was diluted to one-half of the recommended concentration in 1 ml of biotin-free casein colloidal buffer (RDI, Flanders, N.J.). After incubation for 2 hours at room temperature, the solution was discarded and the washing sequence repeated. The membranes were then incubated with 2 ml of the supplied APO diluted 1:20,000 (one half the normal concentration) in the casein blocking solution. After one half-hour the membranes were subjected to the washing sequence.

For comparative analysis, matched sets of samples and control arrays were developed and imaged in tandem. For maximal sensitivity, each of the membranes was incubated with 1 ml of a freshly prepared solution of SuperSignal® West Femto (Pierce) for 1 minute. The membranes were stained, sandwiched between sheets of Saran Wrap and imaged using a hand luminometer (Analytical Luminescence Laboratory, San Diego, Calif.) equipped with Fuji FB-3000B film. Imaging was initiated within 5 minutes of addition of the substrate, with the film serially exposed for varying lengths of time ranging from 10 seconds up to more than one half hour. Imaging continued as the signal decayed providing multiple images. As the signal decayed, the difference between the samples and negative control membrane often became more pronounced. Imaging was also accomplished in a Biorad ChemiDoc XRS image station equipped with an enhanced sensitivity −45° C. cooled-backed 12-bit CCD with a dynamic range >3. Images were acquired using 3 binning at 3 minute intervals with the image summed over a period of as long 30 minutes. It should be emphasized that while use of an imaging station was not mandatory, this produced data that was linear over a broader dynamic range relative to films.

### Evaluation of Data

As the luminescence decayed with time, the distribution and signal-to-noise ratio of the samples and the negative control arrays changed. To carry out analysis, several images of a particular set of assays were photographed, scanned and digitized. These sets of data or the equivalent CCD-acquired data were image-processed to eliminate the background haze, and the pixel intensity x area density of each of the dot ELISA assays was then calculated using Quantity One,™ a proprietary software (Bio-Rad). Whenever possible, the data was normalized against the average density of the five positive controls on a given array, which was arbitrarily set at one. In theory, the presence of a positive signal could be simply ascertained by subtraction of the data on the control membrane. In practice, this was not always possible. In some instances the intensity of background on a given array was not uniform or the intensity of the signals from the positive controls on the top of the membrane differed significantly from those at the bottom of the array. Moreover, this type of analysis was not possible using a standard array where the intensity of luminescence on the positive controls greatly exceeded the linear range of either the film or camera. This type of analysis was also not possible using the capped pre-conditioned membranes. In these situations, arrays were visually analyzed and to ensure the reliability of the data, the results were blindly and independently scaled by three observers and compiled.

While the obtained data was non-quantitative in nature, at times it was used to obtain a crude estimate of the likely concentration range of some of the detected proteins. This estimation was based on the relative ratio of the sensitivities of each of the dot ELISA assays as posted on the manufacturer's web site, the known concentration range established for TIMP-1 and for IL-8 in RTF, OTF and CTF fluids and the relative area intensity ratios of the dot ELISA data obtained for the protein in question and that of TIMP-1 and IL-8. The validity of this analysis is predicated on the assumption that the signal for each of the ELISA assays (minus that on the negative control array) is within the linear range of the assay and specific in nature, can be attributed solely to the probed protein, and that the relative sensitivities of the assays as listed on the manufacturer's web site are accurate.

The specificity and lack of cross-reactivity of each of the ELISA assays found in the array has been determined by the manufacturer using a panel of recombinant standard proteins with the array partially validated for use for serum and urine analysis. This relationship does not necessarily hold for other complex biological fluids such as tears. In evaluating the data of the present invention, it is therefore important to recognize that the invention provides the presence of an antigenic reactive species that most likely represents the probed protein.

### Western Blot Analysis

Western blot analysis was used to confirm the presence and to determine the approximate concentration range of ANG in tear fluid. In this instance RTF, OTF and CTF samples and a serial dilution of recombinant ANG were separated on 16% SDS PAGE mini-gel under reduced conditions and blot transferred onto nitrocellulose following a previously published protocol. Probing was carried out using a goat polyclonal antibody specific for ANG as the primary antibody (antibodies and recombinant ANG purchased from R and D systems Inc. Minneapolis, Minn.). Detection was carried out using an AP-linked anti-goat IgG (Sigma) and NBT/BCIP (Sigma) as a substrate. While it would have been preferred to carry out western blot analysis on several of the other detected tear proteins as well, time and cost constraints made this impossible.

### Results

In a typical assay of a 300 μl RTF sample using the large array kit as configured and imaged with the supplied luminol based substrate, only three signals could be detected. These consisted of an intense signal for ANG (a protein not previously known to be present in tear fluid) and far less intense signals for two known tear constituents, TIMP-2 and TIMP-1. Western blot analysis confirmed that ANG is present in RTF in the form of an 14 kDa species and that it is present in RTF in concentrations in the sub-ng/μl range.

Coupling the array to an ultra-sensitive substrate system and optimizing the assay protocol as contemplated herein greatly enhanced the sensitivity of detection thereby allowing the visualization of positive signals for at least 11 antigenic species in 50-100 μl tear samples obtained from all 6 donors (Table 1) with the intensity of all of these signals markedly higher in the CTF compared to OTF and RTF samples.

Increasing the sample size to several hundred μl of RTF increased the signal-to-noise ratio thereby allowing the tentative identification of numerous additional species with the vast majority present in much higher levels in CTF compared to RTF and OTF fluids.

A further increase in the signal-to-noise ratio was obtained by the partial removal of non-specific interacting species from both the array and the cocktail of biotinylated probe antibodies prior to the assay (as described above) allowing the visualization of upwards of 39 antigenic reactive species in a pooled CTF sample (see Table II). Stripping of the membrane after visualization followed by re-probing with SAP (see above) further improved the signal-to-noise ratio and allowed the visualization of several previously cryptic species. This includes numerous chemokines and leukochemokines (Table III).

In order to explore further the sensitivity limits of this technology, a mini-array was constructed in which the grid geometry was compacted and the array complexity greatly reduced to probe for only 12 proteins. Decreasing the complexity of the cocktail of biotinylated antibodies resulted in a profound reduction in the level of non-specific reactivity on the negative control array thereby markedly increasing the signal-to-noise ratio. This permitted the detection of up to 10 of the 12 probed proteins in 20 μl of CTF. Detected species included trace levels of several entities such as VEGF that could not be detected above the noise using a four-fold larger CTF sample with the standard array. Calibrating the array assay with a known mixture of recombinant protein standards suggested a threshold of sensitivity for some of the assays well into the sub-picogram/ml range. Therefore, the approach of the present invention was most successful when using the small custom array where the problem of high background noise was minimal.

Under conditions in this Example, as many as ten of the twelve probed proteins were detected using as little as 15 μl of CTF. This included several proteins (i.e. VEGF) that were not detectable (due to the high background noise level) using larger pooled tear samples and the standard array. However, using larger pooled samples, with the standard array some proteins such as IL 1β that were not detected in normal tear fluid using standard ELISA assays were detected.

The array in this Example detected positive signals for as many as 40 of the 79 probed proteins in tear fluid. These include many proteins that are bioactive in trace amounts, many of which have never been observed in tear fluid. Also, this Example unequivocally demonstrated a profound difference in the relative distribution of many of these entities in tear samples collected under two distinct physiological conditions in a manner consistent with the different physiological functions of the pre-ocular tear film under open and closed eye conditions. Based upon this finding, array analysis of the present invention can be a useful tool in identifying biomarkers and mediators of ocular surface diseases in tear fluid.

Strong positive signals were consistently obtained for 11 of the 79 probed proteins in all types of tear samples from all donors (Table I). Not surprisingly, the vast majority of these signals came from dot ELISA assays that exhibit a low threshold of sensitivity (exception being TIMP-1). Five of these signals came from proteins (IL-8, EGF, TIMP-1, TIMP-2 and MCP-1) that have been previously documented to be present in tear fluid in concentration ranges that are consistent with the obtained data (In the case of MCP-1, detection has previously been restricted by ELISA assay solely to CTF).

Significantly, the intensity of the signals that are obtained for these and the vast majority of the other detected species were consistently higher in CTF compared to the RTF and OTF samples. In some instances the magnitude of the difference was exponential in nature. This finding is strikingly different from the pattern of distribution that has been established for the three major inducible lacrimal secretory proteins (lysozyme, lactoferrin and tear specific pre-albumin). The latter proteins have been shown to remain relatively constant in tear fluid irrespective of the mode of sample collection. Since this relationship proved true for EGF as well as for trace signals for HGF, two cytokines that have previously been attributed at least in the open eye condition to a lacrimal gland origin, one must conclude that in the closed eye environment, the inducible lacrimal gland secretion is at best only a minor contributing source for the vast majority of bioactive trace tear proteins.

The other six prominent signals represent proteins that have not been previously reported in tear fluid. These include IP-10, GRO (generic), IGFBP-2, ANG and ENA-78. The most surprising of these findings was the very high levels of signals that were seen for ANG in virtually all tear samples.

Array analysis reveals the accumulation in CTF of several members of the CXC family of chemokines—the most prominent being IL-8, ENA-78, IP-10, and GRO, as well as two CC macrophage specific chemokines, MCP-1 and MIP-1beta (Table II).

### EXAMPLE 3

Using the antibody array analysis (AAA) describe in Example 3, the relative distribution of more than 80 growth factors, cytokines, chemokines and angiogenic modulators in openand CTF was characterized. This allowed the identification of a wide range of CXC (some antibacterial) and CC chemokines, including MCP-1, GRO, ENA-78 and NAP-1 that accumulate in CTF. These findings illustrated an extensive epithelial contribution to the closed eye defense mechanism. These factors can also involve in PMN recruitment and enhance the efficiency of SIgA and surfactant D opsonization of entrappedmicroorganism. PMN cell degranulization has been known to result in the accumulation in CTF of toxic reactive products such as MMPs, elastase, cathepsin G. This in turn is balanced by the accumulation of anti-proteases in part derived from the ocular surface tissue.

Many multifunctional antimicrobial agents were identified in tears, many of which accumulate in CTF. These included high concentrations of many small highly basic multifunctional proteins such as angiogenin and SLIPI that are ideally suited to complex with the acidic groups common to the mucosal matrix and interacting with the resident pool of PMN cells.

Angiogenic assays revealed that CTF exhibited net angiogenic activity. Partial purification of the active species illustrated properties consistent with possible new bioactive species, such as IGFBP-2 and neurotrophic growth factors. AAA analysis revealed the accumulation in CTF of high levels of numerous angiogenic modulators. AAA also revealed the presence of high levels of several previously undetected growth factors as well as markedly higher levels of well-known growth factors such as VEFG, EGF and HGF. These findings can demonstrate that the ocular surface and or recruited inflammatory cells rather than the lacrimal gland is the major source of these proteins, many of which were found in bioactive concentrations.

### EXAMPLE 4

The present example assayed proteins consisted of 9 TH-1/TH-2 cytokines, 8 angiogenic modulators and 9 MMP constituents (one redundant with the angiogenic array), each of which are known to modulate inflammatory and immune processes, epithelial cell migration, angiogenesis, apoptosis and differentiation or wound healing in ocular and other tissues. The results demonstrated the usefulness of this technology for tear protein analysis, which can identify biomarkers of KCS.

### Methods

### Subjects and Tear Collection

The tear samples were obtained by two methods. The vast majority of tear samples ranging in size from 3 to 10 μl were slowly collected over a ten to twenty minute period using calibrated glass microcapillary tubes from the lower formix. A minority of tear samples was collected by a novel procedure based upon the adoption of a protocol pioneered by Phuegfelder.

Tears were collected by placing a sterile wick composed of a contact lens polymer (was available from CIBA) on the surface of the lacrimal lake where the wick functioned much as a sponge. Once the bottom was wetted, the wick was sealed inside of an Eppendorf tube, which was kept on dry ice. On thawing, the strip was placed within a disposable plastic pipette tip which rested in an Eppendorf tube and the tear fluid eluted off the wick by centrifugation.

Samples were collected before any ocular examination from 25 individuals who were free of any history or symptom of ocular allergies or ocular surface tissue diseases. Pathological tears samples were recovered from 23 individuals who had been previously diagnosed with non-SS aqueous deficiency dry eye syndrome. The diagnosis and relative severity of the disease was graded based upon clinical symptoms, tear breakup time, Rose Bengal cornea staining, and Schirmer strip evaluation into three categories consisting of those individuals who exhibited mild, moderate and severe dry eye symptoms and pathologies. 8 to 10 μl of tear fluid was collected from the majority of these individuals. In three instances insufficient tear fluid volumes could be recovered from a given individual to allow analysis. In these instances the tear samples were combined from all three individual giving a 12 μl sized sample. After collection the samples were double blind coded transferred to siliconized eppendorf tubes and sent to the laboratory in dry ice and the stored at −78° C. Prior to analysis all the samples were centrifuged (11,000 rpm, 30 minutes, 4° C.) and the supernatants used for assay.

In addition to the assayed basal type tear samples, large volumes of reflex type tear fluid (RTF) was recovered from several normal individuals using nasal stimulation. Most of this fluid was pooled to form a stock solution. This and the remainder of the individual RTF samples were used to prepare spiked tear samples to calibrate the recovery of recombinant protein standards in the presence of RTF. This proved possible for all of the recombinant proteins except for TIMP 1 and 2 and IL-8 which were present in too high concentrations in RTF to allow calibration.

### Microwell Plate Sandwich ELISA Array Assays

Analysis was carried out using three off the shelf antibody array kits (the Pierce SearchLight™ HumanAngiogenic Array, the Pierce SearchLight™ TH1/TH2 cytokine array and the Pierce MMP array) with the individual tear samples assayed by sequential transfer from one array to another in the order listed.

The initial assays were carried out using the Searchlight™ TH-1/TH-2 array a kit which is designed to simultaneously measure IL-2, IL-4, IL-5, IL-8, IL-10, IL-12, IL-13, interferon-gamma (IFNγ) and tumor necrosis factor-alpha (TNFα). The sensitivities of each of the assays are 0.2, 0.4, 0.2, 0.4, 0.2, 0.6, 7.8, 0.2 and 4.7 pg/ml, respectively. After incubation of the tear samples and dilutions of standards ((a combined pooled composites of the recombinant proteins standards from all three of the arrays (see below)) were quantitatively harvested from the respective and transferred to wells on a pre-blocked (see below) Pierce SearchLight™ angiogenic array. This array was designed to allow the simultaneously assay eight proteins, e.g., angiopoietin-2 (ANG-2), vascular endothelial growth factor (VEGF), heparin binding epithelial growth factor (EGF-(1, 4)), basic fibroblast growth factor (bFGF), platelet-derived growth factor-BB (PDGF-BB) (HGF), keratocyte growth factor (KGF), transforming growth factor (TGF), and tissue inhibitor metalloprotease-1 (TIMP-1). After incubation, the residual tear fluids and combined recombinant standards were once again salvaged and quantitatively transferred to a third array specific for matrix metalloprotease (MMP) MMP1, MMP-2, MMP-3, MMP-8, MMP-9, MMP-10, MMP-13 and TIMP-1 (redundant assay) and 2.

All of the utilized arrays are designed to be used in a plug and play basis and as such are sold as kits which include all the necessary reagents and standards to carryout analysis of samples of volumes of up to 50 μls using a methodology that is described is detailed in the supplied instruction manuals available on the manufacturer's Web site. During the course of preliminary studies, it was found that when these arrays were used as directed for tear analysis, two specific matrix effects predominated which rendered these assays useless. Briefly, these matrix effects consist of the non-specific deposition of a highly sticky tear protein(s) or complex which exhibited a marked affinity for the well plastic matrix. This substance(s) subsequently bound the secondary biotinylated antibodies resulting in extremely high background reactivity. The second matrix effect consisted of the partial to complete blocking of the capacity of antigens to the intended capture antibodies. This particular matrix effect phenomenon has also been reported. The assay protocols in the present invention were modified on the basis of preliminary studies to greatly reduce the impact of these artifacts on the reliability of almost all of the 23 ELISA assays. The exception being the assay for ANG-2, which cannot at present be validated in the presence of tear fluid. The following protocol allows the semi-quantification of all of the remaining assayed proteins.

To carryout analysis, a pre-incubation step consisting of incubation in 50 μl of MEGA Block3™ in buffer (a proprietary synthetic blocking agent) for one hour at room temperature was added to the assay protocol. This served to block reactive binding sites which are common to the well plastic surface and thereby greatly decreased the predilection of a highly sticky tear factor(s) to aggregate around the well matrix. The tear samples (8-10 μl) along with a serial dilutions of a combined cocktail consisting of dilutions of all three of the supplied recombinant standards were reconstituted in MEGA Block3™ and the 50 μl volumes were added to each well. The same set of standards were spiked in 10 μl of pooled stock RTF which were added in duplicates to individual set of wells in order to approximate the efficiency of standard recovery in the presence of tear fluid. Preliminary studies were conducted to compare the variability of protein recovery using standards spiked in different volumes of RTF from different individuals. MEGA Block3™ was included as a diluent to all of these samples since it greatly increased the capacity to recovery recombinant protein standards in spiked tear samples. Due to the sample size limitation and the relative lack of sensitivity of some of the assays, most of the pathological tear samples contained sufficient volumes to carryout only a single assay although occasionally sufficient volumes were present to allow the assay in duplicate. In most of the normal tear samples, assays were carried out in duplicate in two dilutions. After incubation for 30 minutes at room temperature, the residual fluid in the samples and the standard wells are quantitatively harvested and transferred along with a 5 μl wash from each well to a pre-incubated angiogenic array and the incubation process repeated. After this incubation the samples were transferred to a third pre-blocked array specific for MMP proteins. To obtain information on the likely degree of loss of protein from the multiple transfer process, several wells on the angiogenic and MMP arrays contained fresh solutions of protein standards.

After 30 minutes incubation at room temperature with agitation, the wells were emptied, washed five times with a supplied washing buffer and replaced with 50 μl of a cocktail of the biotinylated secondary antibodies in blocking buffer. The plate was incubated with agitation at room temperature as directed. The wells were again emptied and the washing sequence was repeated. The wells were then incubated for 30 minutes with 50 μl of a supplied solution of streptavidin-linked peroxidase (SPO) reporter enzyme in blocking buffer. After discarding this fluid, the wells are subjected to a final series of washes.

Within ten minutes of the addition of the supplied luminol-based enhanced sensitive substrate (e.g., Femtogram SuperSignal™ from Pierce), detection was carried out by imaging using a Chemdoc XRS image station (Biorad) equipped with a deep cooled CCD camera. Imaging was carried out without binning, using the supplied substrate for periods of one to ten minutes or over a several hour period with background noise subtracted when using ChemiGlow™. The images of each of the wells in the plate were visually examined for artifactual background chemoluminescence before densitometric analysis. In the few instances where no specific deposition occurred this was restricted primarily to the well edges. In these instances the data from affected nearby dot ELISA assays were either excluded from analysis or when possible processed with Quantity one-4.5.0™ (Biorad proprietary software). This allowed for background subtraction. Using the same software, two sets of standard curves were constructed: one based upon the data obtained from analysis of serial dilutions of neat recombinant protein standards and the other based upon the data obtained using spiked pooled RTF. The protein concentrations in the samples were estimated by extrapolating the standardizations curves from the spiked samples. This approach as mentioned was not possible in the cases of TIMP-1, TIMPs-2 and IL-8 where the high base line levels of these proteins in RTF exceeded the linear range of the assay.

### Results

The results showed the profound impact of two matrix effects (aggregation and blocking) on the capacity to carryout to ELISA assays for TH-1/TH-2 cytokines using the Pierce array kit as proscribed by the manufacturer.

The result showed a set of two dilutions of the other recombinant proteins standards neat and spiked in a stock RTF matrix using the modified protocol of the present invention as described above.

The results reveal a suppression of both matrix effects and the capacity to recovery approximately 50 to 80% of each of the recombinant protein standards. The extent of quenching of the signal varies with the source and volume of the spiked tear fluid, making the obtained data semi-quantitative in nature.

Assay of the normal and the dry eye tear obtained samples revealed a very similar pattern of cytokine distributions. Other than increased level of IL-8 in many of the dry eye tear samples, only trace levels of the other probed cytokines could be detected in both the pathological and normal tear samples. The result showed that normal OTF contains high levels of IL-8 and at most trace levels of other measured TH-1/TH-2 cytokines (IL-4, TNF). Overnight eye closure is associated with a massive build up of IL-8 and an increase in the concentrations of many other cytokines in tears. Chronic allergic reactions associated with elevated levels of IL-2, 4, 5, 8, 10, INF and TNFa with this increase most pronounced in CTF.

Comparative assays of the recombinant protein standards neat and in spiked RTF reveals widely divergent patterns in the degree to which the addition of RTF quenched the signal of the recombinant proteins. Particularly dramatic was the total loss of signal for ANG-2 and an inverse pattern of ANG-2 detection on sample dilution made the assay for this protein impractical.

Far better results were obtained for the remaining 6-targeted proteins with the percent recovery of TIMP-1 not quantifiable due to the very high levels of this protein in the RTF. Comparative analysis of the normal and dry eye tear samples revealed a marked difference in the concentration and pattern of distribution of many of these entities in a subgroup of the pathological tear samples. The normal tear samples invariably contained exceptionally high levels of TIMP-1 and often-variable levels of VEFG and HFG with other entities only occasional detected in minute quantities. In contrast, the majority of the dry eye obtained tear samples in addition to these entities contained anywhere from low to very high levels of FGFb and Hb-EGF growth factors not detectable in the normal tear fluid.

Assay of the recombinant proteins standards in net and spiked tear samples using the MMP array revealed a partial variable degree of quenching of the protein standards with quenching particularly evident in the case of MMP-9. The percentage of the recovery of TIMPs 1 and 2 could not be ascertained in the spiked sample due to the exceptionally levels of these proteins in the RTF. Comparative analysis of normal and pathological reveals marked different pattern and variable patterns in the distribution of MMPs in the normal and the majority of the pathological tear samples. In many samples keratosicca samples the levels of MMP 2, 3, 8, 9 and 10 were decidedly higher than that in the normal tear samples.

### Discussion

To the best of the inventor's knowledge, this is the first time that microwell plate formatted protein array technology has been employed in tear analysis. The format offers an unparalleled vantage for the simultaneous assay of a small volume of tear fluid for a multitude of LAPs while at the same time allowing for the screening of each of the assays for tear matrix effects which might not be evident using other methods of analysis. As shown above, these matrix effects can profoundly influence the reliability and validity of analysis. Two tear specific matrix effects were evident: the consistent of high non-specific background deposition and the partial to complete blockage of the capacity of the targeted protein to bind the capture antibody. The former effect can be attributed the aggregation of a highly sticky tear factor on the plastic matrix. This effect subsequently complexes with secondary antibody thereby resulting high background reading. Blockage in turn can be attributed to the presence in tear fluid of various blocking factors. The failure to recognize and adequately account for these matrix effects could well be a factor in the hundred fold or more difference that has been reported for the levels of various cytokines, chemokines and growth factors in tear fluids in earlier studies.

The above example of present invention greatly minimized the impact of these effects by altering the assay protocol thereby allowing the assay of most of the probed targeted proteins. Significantly, in the case of ANG-2, assay in tear fluid was not feasible as demonstrated the total loss of the recombinant protein standard in the spiked tear fluid. These findings certainly highlight the necessity to always check the validity and accuracy of any ELISA type assay by carrying out standardization with spiked tear samples.

In other assays, the recovery of recombinant proteins varied anywhere from approximately 50 to 80% of the neat protein standard. At present it is uncertain to what extent the efficiency of recovery or the prevalence blocking varies from tear sample to sample or with the type of tear sample (open, reflex or closed). This coupled with the fact that the small size of many of the dry eye samples precluded multi assay results in data which at best should viewed as semi-quantitative in nature.

The present example increased the breath of assay by sequentially transferring the same samples through three arrays. While larger arrays are available which allow the simultaneous dot ELISA assay of as many of 36 proteins per well, increasing the array size has the decided disadvantages. As the array size is increased the signal to noise ratio of individual assays often is impaired due to an enhanced level of cross talk between the capture antibodies and the cocktail of secondary antibodies. Moreover, it becomes increasingly more difficult to design arrays in which all of the probed proteins in a biological sample lie within the linear range of all of the assays. In theory, these problems can be circumvented by passage of the same sample through multiple arrays. In the present invention, the quality of the obtained data could have been further improved by utilizing custom configured and validated arrays which would segregate and compartmentalize the assays of IL-8, TIMPs-1 and 2 into a separate array. These proteins could be assayed with much smaller tear samples (<1 μl) thereby bring data within the linear range of these assays and eliminating the redundancy for the assay of TIMP-1. Budgetary and time constraints, however, precluded this possibility. Irrespective of these limitations, the obtained data serves as a proof of principal and provides a wealth of data on the relative distribution of a wide range of bioactive LAP in normal and dry eye tear fluid. Some of these entities can participate in the pathophysiological processes.

Particularly striking is the marked difference in the pattern of distribution of various angiogenic modulators and MMPs in the normal and pathological tear samples. FGFb and Hb-EGF which are virtually absent or barely detectable in virtually all of the normal tear samples are very prominent entities in many of the dry eye tear samples, reaching concentrations which at times approaches the ng/μl range. That these growth factors are absent, or found at most in trace levels, in normal tear fluid confirms the findings of previous reports. The finding of a very marked increase in Hb-EGF in the pathological tear samples is surprising since the level of EGF was reported to be lower in tear fluid from individuals with both SS and non-SS aqueous deficiency dry eye syndromes. The result of the present example illustrates that Hb-EGF and EGF two closely related growth factors exhibit an inverse pattern of regulation. All of these growth factors, are known to be synthesized by corneal epithelium, keratocytes, endothelium, and the lacrimal gland. Much less is known about the conjunctiva. Hb-EGF is known to secreted and bound to the corneal epithelium and other epithelial where it is found complexed with glycoproteins on the cell membrane through its heparin-binding domain. Several MMPs and other proteases are known to clip the glycoprotein releasing free HP-EGF from the cell membrane. This can be the source of the marked increase in HB-EGF in the pathological tear samples. HB-EGF in turn is known to bind to the EGFr. Chronic allergic reactions are associated with an exponential increase in the concentrations of FGFb and Hb-EGF in tear fluid. It is believed that HB-EGF can be derived from HB-EGF normally bound to the epithelial cell membrane. This could be cleaved by various ADAM-like proteases including MMP-3. Released Hb-EGF could modulate apoptosis, cell migration and turnover through binding to the EGF receptor. FGFb could up regulate wound healing through stimulation of keratocytes.

It is of interest to note that virtually all tear samples exhibited a detectable signal for stromolysin 3 (i.e., MMP-10) and that the signal is elevated in many of the dry eye tear samples. This was often companied by the detection of stromolysin (i.e., MMP-3) which cannot be detected in the normal tear sample. To the best of inventors' knowledge this is the first time that MMP-10 has been reported in normal tear fluid with the presence of MMP-3. MMP-3 was previously detected only in tears from individuals with active acne rosaeia. Pflugfelder's laboratory has presented data that strongly suggests that MMP-10 participates in the pathophysiological process in acne rosaeia by cleaving and activating proMMP-9. MMP-9 and other disintegrins, such as ADAM-12, that are known to cleave the epithelial bound HB-EGF complex releasing HB-EGF. The recognition of the present invention certainly supports the concept of a functional role for MMPs in the pathophysiology of dry eye syndrome.

The presence of a strong signal for MMP-8 which is specific for neutrophiles in many of the dry eye tear samples supports the premise of the recruitment and activation of PMN cells in this processes. The result supports an earlier report from the inventor's laboratory of elevated levels of neutrophil associated lipocalin (NGAL) in tear fluid from individual experiencing dry eye type symptomology. This finding is also compatible with recently reported of SELDI-TOF-MS that two of seven protein biomarkers associated with KCS consist of calgranulin A (a neutrophil specific protein) and the C terminal fragment of alpha1 anti-trypsin. The inventor has previously shown that alpha-1-antitrypsin is one of the major serpins that is present in tear fluid; that its concentration increases markedly in tear fluid during overnight eye closure; that its concentration appears to be upregulated in response to the build up of PMN cell proteases; and that it rapidly reacts in the tear fluid with PMN cell derived elastase and proteinase-3 giving rise to protease-antiprotease complexes and the C-terminal fragment. Taken in total this data strongly suggest an active participation of PMN in at least a sub group of the individuals with KCS symptoms.

Hb-EGF is known to bind to the EGFr and thereby modulate epithelial cell function by regulating apoptosis and cell turnover. Thus one can postulate that that the presence of high levels of Hb-EGF suggestion a shift in epithelial turnover. These pathways are unlikely to be unique to the patholophysiology of dry eye and may instead be representative of a common down stream process common to other surface conditions.

TABLE 1
Major antigenic species detected in all RTF samples

| Protein | Relative Sensitivity of Assay | Corroborating Data |
|---|---|---|
| ANG | 10 | wb |
| EGF | 1 | |
| ENA-78* | 1 | — |
| GRO | 1 | — |
| IL-8 | 1 | wpma, wb, |
| MCP-1 | 3 | |
| IGFBP-2 | 10 | |
| IP-10 | 10 | — |
| TIMP-1 | 100 | wpma, wb, |
| TIMP-2 | 1 | wb, |
| MIP-1b | 10 | — |

wb western blot,
wpma well plate membrane array assay,
*variable;

TABLE 2
Proteins detected in closed eye tear fluid (CTF) by MA

| | relative | high | intense | moderate | faint | 75-100% | 50-75% |
|---|---|---|---|---|---|---|---|
| Protein | sensitivity | background | signal | signal | signal | samples | samples |
| ANG | 10 | | x | | | x | |
| EGF | 1 | | x | | | x | |
| ENA-78 | 1 | x | | x | | x | |
| EOTAXIN | 1 | | | x | | x | |
| EOTAXIN-2 | 1 | | | x | | | x |
| GCSF | 2000 | | | | x | x | |
| GDNF* | 100 | x | | | x | x | |
| GM-CSF | 100 | | | | x | x | |
| GRO | 1 (gamma) | | x | | | x | |
| GROa | 1000 | | | | x | x | |
| HGF | 200 | | | | x | | x |
| IFN-γ | 100 | | | | x | x | |
| IGFBP-1 | 1 | | | | x | x | |
| IGFBP-2 | 10 | | x | | | x | |
| IL-6 | 1 | | | x | | x | |
| IL-8 | 1 | | x | | | x | |
| IL-10 | 10 | | | | x | x | |
| IL-12 | 1 | | | | x | | x |
| IL-15 | 100 | | | | x | | x |
| IL-16 | 1 | | | | x | | x |
| IP-10 | 10 | | x | | | x | |
| MCP-1 | 3 | | x | | | x | |
| MCP-2 | 100 | x | | x | | | x |
| MCSF | 1 | | | | x | | x |
| MDC | 1000 | | | | x | | x |
| MIP-1b | 10 | x | | x | | x | |
| NAP-2* | 100 | x | | x | | x | |
| NT-3 | 20 | | | | x | | x |
| NT-4 | 2 | | | | x | | x |
| TGF-b2* | 1000 | x | | | x | x | |
| TIMP-1 | 100 | x | x | | | x | |
| TIMP-2 | 1 | | x | | | x | |
| TNF-a | 10 | | | | x | x | |

*uncertain findings because of high background and/or faint signal

TABLE 3
Chemotactic, angiogenic and antibacterial characteristics
of CXC-Chemokines present in tears

| | Chemotactic | Angiogenic | Antibacterial |
|---|---|---|---|
| Protein | properties | properties | properties |
| IL-8 | Np, T, B, Ba, EC | + | − |
| GRO | Np, Ba, EC | + | − |
| NAP-2 | Np, EC | + | − |
| ENA-78 | Np, EC | + | − |
| GROα | Np, T, B, Ba, EC | + | − |
| IP-10 | Tac, M, NK, EO | angiostatic | + |
| MIG | Tac, EO | angiostatic | + |

B B-lymphocytes,
T T-lymphocytes,
Tac activated T-lymphocytes,
EC Endothelial cells,
Np Neutrophil granulocytes,
Ba basophil granulocytes,
Eo eosinophil granulocytes,
M Monocytes,
NK Natural killer cells;

What is claimed:

1. A method for simultaneously identifying low abundance proteins (LAPs) in a tear fluid sample comprising the steps of a) obtaining the sample, b) incubating an antibody-based stationary phase array with a blocking buffer, c) incubating the sample from a) with the array from b), d) incubating the array from c) with detection/secondary antibodies, e) incubating the array from d) with an ultra-sensitive substrate that is reacted with an enzyme linked to the detection antibodies thereby providing a detectable signal of the binding between a capture antibody on the array and a LAP, f) detecting the signal.

2. The method of claim 1, wherein the LAP is selected from the group consisting of matrix metalloprotease (MMP), angiogenin (ANG), HGF, FGFb, TPO, VEGF, KGF, HB-EGF and PDGF-BB, interleukins (ILs), interferons (IFNs) and TNF, and TIMP.

3. A method for diagnosing a pathological condition in a subject comprising the steps of a) obtaining a biological fluid sample from the subject, b) identifying the LAPs in the sample by the method of claim 1, and c) detecting and analyzing the changes of the LAPs in the sample relative to that of a normal sample or to a database comprising known LAP distribution/level patterns under normal or pathological conditions.

4. The method of claim 3, wherein the pathological condition is an ocular disease and wherein the sample is a tear sample.

5. The method the claim 4, wherein the ocular disease is an ocular infection or ocular inflammation.

6. A method for treatment of ocular infections and/or inflammation in a subject, comprising a) detecting or diagnosing an ocular microbial infection in the subject according to claim 3, and b) administering one or more anti-microbial agents to the subject.

7. A method for prevention of ocular infections and/or inflammation in a subject, comprising a) detecting or diagnosing angiogenin (ANG) level in a tear fluid sample from the subject, b) comparing ANG level of the sample with a normal reference range of ANG from tear samples, and c) if the ANG level is lower than its normal range, administering ANG and/or at least one other anti-microbial agent.

8. A method for differential screening/analysis of LAPs in tear samples obtained from different physiological conditions, comprising the steps of a) obtaining the samples, and b) identifying and comparing/analyzing the LAPs in each sample.

9. An antibody-based stationary phase array system comprising an array matrix of dot grids on a stationary phase bounded by at least one antibody that is capable of binding with a specific protein species, at least one secondary or detection antibody, and an ultra-sensitive substrate that is recognized by an enzyme linked to the secondary antibodies.

10. A kit for diagnosing ocular pathological conditions comprising an instruction manual, an antibody-based membrane array, a reaction-well tray, blocking and washing buffer solutions, detection antibodies, at least one indicator that detects a specific binding of trace proteins in a test sample to the capture antibody or antibodies carried by the array.

11. A kit containing a composition in the form of eye drops for anti-ocular microbial infection, comprising recombinant angiogenin and a pharmaceutically acceptable carrier.
